(19) United States
(12) Patent Application Publication
(10) Pub. No.: US 2005/0113409 A1
(43) Pub. Date: May 26, 2005

(54) Method for the prevention or treatment of pain, inflammation and inflammation-related disorders with a Cox-2 selective inhibitor in combination with a nitric oxide-donating agent and compositions therewith

(75) Inventors: Jane R. Connor, University City, MO (US); Pamela T. Manning, Chesterfield, MO (US)

Correspondence Address: Charles E. Dunlap;Nelson Mullins Riley & Scarborough, LLP, 17th Floor, 1320 Main Street, Columbia, SC, 29201, US

(73) Assignee: Pharmacia Corporation, Chesterfield, MO (US)

(21) Appl. No.: 10/932,385

(22) Filed: Sep. 2, 2004

Related U.S. Application Data
(60) Provisional application No. 60/499,861, filed on Sep. 3, 2003.

(51) Int. Cl. A61K 31/47; A61K 31/382; A61K 31/353; A61K 31/277; A61K 31/18

(52) U.S. Cl. 514/311; 514/432; 514/456; 514/602; 514/520

(57) ABSTRACT

A method is described for the prevention or treatment of pain, inflammation, and inflammation-related disorders in a subject in need of such prevention or treatment, the method comprising administering to the subject a cyclooxygenase-2 selective inhibitor in combination with a nitric oxide-donating agent. Also described are therapeutic and pharmaceutical compositions and kits that are useful in the present invention.

### CROSS REFERENCE TO RELATED PATENTS AND PATENT APPLICATIONS

The subject matter of the present invention is related to and claims priority from U.S. Provisional Patent Application No. 60/499,861, filed Sep. 3, 2003, which is incorporated herein by reference in its entirety.

### BACKGROUND OF THE INVENTION

(1) Field of the Invention

The present invention relates generally to methods and compositions for preventing or treating pain, inflammation, and inflammation-related disorders, and more particularly to methods and compositions related to a combination of a cyclooxygenase-2 inhibitor in combination with another drug.

(2) Description of the Related Art

Inflammation is a manifestation of the body's response to tissue damage and infection. The inflammatory reaction is initiated by the non-specific mechanisms of phagocytosis and complement activation. Although the complex mechanisms of inflammation are not fully elucidated, inflammation is known to have a close relationship with the immune response and to be associated with pain and fever in the subject.

Prostaglandins are known to be important mediators of inflammation, as well as regulators of other significant, non-inflammation-related, functions. A pathological role for prostaglandins has been implicated in a number of human disease states including rheumatoid arthritis and osteoarthritis, pyrexia, asthma, bone resorption, cardiovascular diseases, dysmenorrhea, premature labor, nephritis, nephrosis, atherosclerosis, hypotension, shock, pain, cancer, and Alzheimer's disease. Regulation of the production and activity of prostaglandins has been a common target of anti-inflammatory drug discovery activities. However, common non-steroidal anti-inflammatory drugs that are active in reducing the prostaglandin-induced pain and swelling associated with the inflammation process can also have adverse effects on other prostaglandin-regulated processes not associated with the inflammation process.

Many common anti-inflammatory agents, such as aspirin and ibuprofen, fall under the classification of non-steroidal anti-inflammatory drugs (NSAIDs). It is now widely recognized that many of the traditional NSAIDs are inhibitors of two cyclooxygenases, cyclooxygenase-1 (Cox-1) and cyclooxygenase-2 (Cox-2). These two enzymes are involved in the critical initiation step of prostaglandin synthesis—the addition of molecular oxygen to arachidonic acid in the cell membrane. See Needleman, P. et al., Annu. Rev. Biochem, 55:69-102 (1986).

Cox-1 is constituitively active and is responsible for the synthesis of housekeeping prostaglandins critical to maintaining normal renal function, gastric mucosal integrity, and vascular homeostasis. Cox-2 expression is induced by cytokines and growth factors in inflammatory cells, leading to the release of prostanoids, for example, prostaglandin E2, which sensitize peripheral nociceptor terminals and produce localized pain hypersensitivity, inflammation, and edema. See e.g. Samad, T. A. et al., Nature 410:471-5 (2001). Because many common NSAIDs inhibit prostaglandin synthesis by blocking the activity of both Cox-1 and Cox-2, side effects associated with long-term administration of these drugs such as gastrointestinal bleeding and ulcers are thought to be a result of inhibiting the homeostatic functions of Cox-1, while the inhibiton of Cox-2 accounts for their analgesic properties.

Research into the area of arachidonic acid metabolism has resulted in the discovery of compounds that inhibit the cyclooxygenase-2 enzyme to a greater extent than the activity of cyclooxygenase-1. The Cox-2-selective inhibitors are believed to offer advantages that include the capacity to prevent or reduce inflammation while avoiding harmful side effects associated with the inhibition of Cox-1. Thus, Cox-2 selective inhibitors have shown great promise for use in therapies, especially in therapies that require maintenance administration.

Nitric oxide (NO) is a major paracrine signaling molecule in the nervous, immune, and circulatory systems. NO is produced by endothelial cells and is involved in the regulation of vascular tone, platelet aggregation, neurotransmission and immune activation. See Furchgott, R. F. and Zawadzki, J. V., Nature 288:373-6 (1980); Moncada, S., et al., Pharmacol. Rev. 43:109-42 (1991); Ignarro, L. J., Blood Vessels 28:67-73 (1991). Nitric oxide is an important mediator of relaxation of vascular smooth muscle. Nitric oxide is synthesized by the oxidative deamination of a guanidino nitrogen of L-arginine by at least three different isoforms of a flavin-containing enzyme, nitric oxide synthase (NOS). Nitric oxide elevates levels of cGMP (1,4,5-cyclic guanosine monophosphate) within vascular smooth muscle to produce relaxation and to reduce blood vessel tone. Nitric oxide binds to heme and thus activates soluble guanylate cyclase resulting in an increase in cellular cGMP. cGMP activates a cGMP-dependent kinase (PKG), which mediates the vasorelaxant effects of NO by phosphorylating several proteins that regulate intracellular Ca²⁺ levels, effectively reducing cytoplasmic calcium concentration, resulting in vasorelaxation. See Soff, G. A., et al., J. Clim. Invest. 100:2580-7 (1997).

The role that NO plays in the inflammatory response is less well understood. While it is evident that NO production frequently accompanies inflammatory states, it is unclear whether NO promotes, inhibits, or has no effect on the inflammatory process. One mechanism by which NO may influence the progression of the inflammatory process is through its interaction with the NF-κB family of transcription factors. NF-κB regulates the expression of many inducible inflammatory genes, including pro-inflammatory cytokines and endothelial cell adhesion molecules (ECAMs). See Ghosh, S., et al., Annu. Rev. Immunol. 16:225-60 (1998). However, the effect of NO on NF-κB activation is not well understood, and the evidence has been contradictory. It has been demonstrated that NO has an inhibitory effect on both the activation of NF-κB and the binding of NF-κB to its consensus DNA binding site in target genes. See Park, S. K. et al., Biochem. J. 322:609-13 (1997). Also, NO-donors can inhibit TNF-α-mediated activation of NF-κB, suggesting an anti-infammatory function of NO. See Peng, H. B. et al., J. Biol. Chem. 270:14214-9 (1995); see also Peng, H. B. et al., J. Biol. Chem. 270:17050-5 (1995). There is also evidence that NO inhibits the expression of several pro-inflammatory genes, including chemokines, adhesion molecules, interleukins, and cyclooxygenase-2. See Colasanti, M. and Persichini, T., Brain Res. Bull. 52:155-61 (2000). However, there is also a wealth of data suggesting that NO has pro-inflammatory actions. See Laroux, F. S., et al., Acta Physiol. Scand. 173:113-8 (2001).

Given the fact that NO plays a role in such a variety of important bioregulatory processes, great effort has been expended to develop compounds capable of releasing NO. Some of these compounds are capable of releasing NO spontaneously, for example, by hydrolysis in aqueous media, whereas others are capable of releasing NO only upon being metabolized. See Lefer et al., Drugs Future 19:665-672 (1994).

It has long been recognized that nitrovasodilators, such as nitro-prusside and nitroglycerin, inhibit vascular smooth muscle contractility to produce relaxation or to reduce vascular tone. These agents have been used since the late 1980s as vasodilators. However, only recently has the mechanism of action of these compounds been realized. Nitrovasodilators are now classified as nitric oxide donors because they are metabolized to, or spontaneously release, nitric oxide.

There is little known about the effects of NO on Cox-2 inhibitor-mediated anti-inflammatory actions. Several U.S. Patents describe the addition of NO or NO₂ groups to known NSAIDs in order to decrease the gastrointestinal and renal side effects of this class of anti-inflammatory compounds. These patents include U.S. Pat. No. 6,593,347 to Bandarage et al., which describes the usefulness of nitrosated and nitrosylated NSAIDs alone or in combination with a compound that releases nitric oxide, as well as U.S. Pat. Nos. 6,359,116 and 6,359,182, both to Stamler et al. Several references include both a cyclooxygenase inhibitor and an NO-donating agent in formulations containing additional components. U.S. Pat. Nos. 5,800,385 and 5,858,017, both to Demopulos et al. disclose surgical irrigation solutions comprising a plurality of agents for direct application to a surgical procedural site. U.S. Pat. No. 5,811,416 relates to combinations of a progestin, an estrogen, a cyclooxygenase inhibitor, and a nitric oxide donor for treatment of menstrual disorders. U.S. Pat. No. 5,910,482 is directed to combinations of calcitonin gene related peptide, a progestin, an NO donor, and a cyclooxygenase inhibitor such as aspirin for the treatment or prevention of preeclampsia.

While it is widely recognized that Cox-2 selective inhibitors are useful in the treatment of inflammation, the effect of a combination of a Cox-2 inhibitor and an NO-donating agent on inflammation and inflammation-related disorders is not known.

Although novel therapeutics, such as Cox-2 selective inhibitors, have been useful as anti-inflammatory agents, there still exists a need for more effective therapies for the prevention and treatment of pain, inflammation, and inflammation-related disorders.

### SUMMARY OF THE INVENTION

Briefly, therefore, the present invention is directed to a novel method for preventing or treating pain, inflammation, and inflammation-related disorders in a subject in need of such prevention or treatment, the method comprising administering to the subject a Cox-2 selective inhibitor in combination with a nitric oxide-donating agent.

The present invention is also directed to a novel composition comprising a Cox-2 selective inhibitor and a nitric oxide-donating agent.

The present invention is also directed to a novel pharmaceutical compostion comprising a Cox-2 selective inhibitor, a nitric oxide-donating agent, and a pharmaceutically acceptable excipient.

The present invention is also directed to a novel kit, the kit comprising a first dosage form comprising a Cox-2 selective inhibitor and a second dosage form comprising a nitric oxide-donating agent, in quantities which comprise a therapeutically effective amount of the combination of the compounds for the treatment or prevention of pain, inflammation, or inflammation-related disorders.

Several advantages are achieved by the present invention, including the provision of an improved method and compositions that prevent or treat pain, inflammation, or inflammation-related disorders, and also a method and a composition that are efficacious for such applications in physiologically acceptable dosages, and which are selective in their physiological impact.

### DETAILED DESCRIPTION OF THE PREFERRED EMBODIMENTS

In accordance with the present invention, it has been discovered that pain, inflammation, and inflammation-related disorders may be treated or prevented in a subject that is in need of such treatment or prevention by administering to the subject a Cox-2 selective inhibitor in combination with one or more nitric oxide-donating agents.

In one embodiment, the method of treating or preventing pain, inflammation, and inflammation-related disorders of the present invention comprises administering to a subject that is in need of such treatment or prevention an amount of a Cox-2 selective inhibitor, which, in combination with an amount of a nitric oxide-donating agent, provides an amount of the combination that is effective for the treatment of the pain, inflammation, and inflammation-related disorder. The effective amount of the combination is preferably a therapeutically effective amount.

As used herein, an “effective amount” means the dose or effective amount to be administered to a subject and the frequency of administration to the subject which is readily determined by one or ordinary skill in the art, by the use of known techniques and by observing results obtained under analogous circumstances. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the subject to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the prevention or treatment”, and both are intended to qualify the amount of each agent for use in the combination therapy which will achieve the goal of improvement in the severity of a cardiovascular disorder and the frequency of incidence over treatment of each agent by itself, while avoiding adverse side effects typically associated with alternative therapies.

In preferred embodiments of the present method, the combination of the Cox-2 selective inhibitor and nitric oxide-donating agent provides results that are unexpectedly superior to those that would have been expected of the combination on the basis of the results obtained by administering either of the two components of the combination alone. For example, in preferred embodiments, the combination of a Cox-2 selective inhibitor and a nitric oxide-donating agent provides therapeutic efficacy at dosages that are lower than those that would normally be expected to be required to produce the same effect based on the results obtained by the use of either agent alone. Such unexpected superiority can also take the form of lower incidence or severity of undesirable side effects, or increased tolerance levels, or improved results as measured by any other commonly used parameter of drug usefulness, safety, or efficacy.

In one embodiment, the present invention encompasses a method for preventing pain, inflammation, and inflammation-related disorders in a subject that is in need of such prevention, the method comprising administering to the subject a Cox-2 selective inhibitor and a nitric oxide-donating agent.

As used herein, the term “prevention” refers to any reduction, no matter how slight, of a subject's predisposition or risk for developing pain, inflammation, or an inflammation-related disorder. For purposes of prevention, the subject is any subject, and preferably is a subject that is at risk for, or is predisposed to, developing pain, inflammation, or an inflammation-related disorder.

As used herein, a subject that is “predisposed to” or “at risk for,” both of which are used interchangeably herein, includes any subject with an increased chance for developing pain, inflammation, or an inflammation-related disorder. The subject may be at risk due to genetic predisposition, diet, age, and the like. The subject may also be at risk due to physiological factors such as anatomical and biochemical abnormalities and certain autoimmune diseases.

In another embodiment, the present invention encompasses a method for treating pain, inflammation, and inflammation-related disorders in a subject that is in need of such treatment, the method comprising administering to the subject a Cox-2 selective inhibitor and a nitric oxide-donating agent.

The terms “treating” or “to treat” mean to alleviate symptoms, eliminate the causation of symptoms, either on a temporary or permanent basis, or to alter or slow the appearance of symptoms. The term “treatment” includes alleviation of, or elimination of causation of, symptoms associated with any of the diseases or disorders described herein.

The term “subject” for purposes of treatment includes any vertebrate. Preferably, the vertebrate is a human or animal subject who is in need of prevention or treatment of pain, inflammation, or an inflammation-related disorder. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human. A subject “that is in need or prevention or treatment”, is a subject who, by genetics, lifestyle, age, physical condition, accident, medical treatment, medical history, or otherwise, is at risk for contracting, or who has contracted, pain, inflammation, or an inflammation-related disorder.

One component of the present invention is a Cox-2 inhibitor.

Inhibitors of the Cox pathway in the metabolism of arachidonic acid may inhibit enzyme activity through a variety of mechanisms. By way of example, the Cox-2 inhibitors used in the methods described herein may block the enzyme activity directly by binding at the substrate site of the enzyme. In preferred embodiments, the use of a Cox-2 selective inhibitor is highly advantageous in that it minimizes the gastric side effects that can occur with non-selective non-steroidal anti-inflammatory drugs (NSAIDs), especially where prolonged treatment is expected.

The terms “cyclooxygenase-2 inhibitor”, or “Cox-2 inhibitor”, which can be used interchangeably herein, embrace compounds, which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, mixed isomer, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, nitroflurbiprofen, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, a 4-(nitrooxy)butyl ester, and mixtures thereof.

Further preferred NSAID compounds include ibuprofen, naproxen, sulindac, ketoporfen, fenoprofen, tiaprofenic acid, suprofen, etodolac, carprofen, ketrolac, piprofen, indoprofen, salicylic acid, flurbiprofen, and mixtures thereof.

In a preferred embodiment, the Cox-2 inhibitor is a Cox-2 selective inhibitor. The term “Cox-2 selective inhibitor” embraces compounds, which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of Cox activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

In one embodiment of the present invention, the Cox-2 selective inhibitor is of the chromene/chroman (“chromene”) structural class, which encompasses substituted benzopyrans or substituted benzopyran analogs, as well as substituted benzothiopyrans, dihydroquinolines, or dihydronaphthalenes having the structure of general Formula I, shown below, and including, by way of non-limiting example, the chromene compounds described below, and the diastereomers, enantiomers, racemates, tautomers, salts, esters, amides and prodrugs thereof.

Chromenes that can serve as a Cox-2 selective inhibitor of the present invention include any one or more of the compounds that are described in U.S. Pat. Nos. 6,271,253; 6,492,390; 6,034,256 and 6,077,850. One such class of compounds is defined by the general formula shown below in formula I: 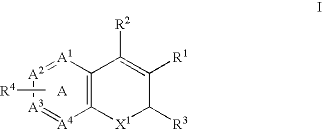
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a cycloalkyl ring;
wherein R¹ is selected from carboxyl, alkyl, aralkyl, aminocarbonyl, C₁-C₆-alkylsulfonylaminocarbonyl and alkoxycarbonyl;
wherein R² is selected from hydrido, phenyl, thienyl, C₂-C₆-alkynyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, phenyl, cyano, cyano-C₁-C₃-alkyl, haloalkyl, alkyl, aralkyl, cycloalkyl, and aryl, wherein haloalkyl, alkyl, aralkyl, cycloalkyl, and aryl each is independently optionally substituted with one or more radicals selected from alkylthio, nitro and alkylsulfonyl;
wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁₋C₆-alkoxy, methylenedioxy, C₁-C₆-alkylthio, C₁-C₆-alkylsulfinyl, O(CF₂)₂O—, aryloxy, arylthio, arylsulfinyl, heteroaryloxy, aralkyloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon; or
wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes a compound having the structure of formula II: 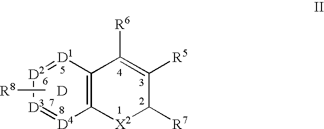
wherein X² is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R⁵ is selected from carboxyl, aminocarbonyl, C₁-C₆-alkylsulfonylaminocarbonyl and C₁-C₆-alkoxycarbonyl;
wherein R⁶ is selected from hydrido, phenyl, thienyl, C₂-C₆-alkynyl and C₂-C₆-alkenyl;
wherein R⁷ is selected from C₁-C₃-perfluoroalkyl, chloro, C₁-C₆-alkylthio, C₁-C₆-alkoxy, nitro, cyano and cyano-C₁-C₃-alkyl;
wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the D ring atoms D¹, D², D³ and D⁴ are independently selected from carbon and nitrogen with the proviso that at least two of D¹, D², D³ and D⁴ are carbon; or
wherein R⁸ together with ring D forms a radical selected from naphthyl, quinolyl, isoquinolyl, quinolizinyl, quinoxalinyl and dibenzofuryl;
or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is: 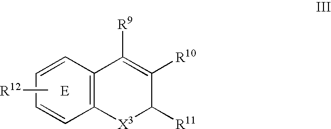
wherein X³ is selected from the group consisting of O or S or NR^{a};
wherein R^{a} is alkyl;
wherein R⁹ is selected from the group consisting of H and aryl;
wherein R¹⁰ is selected from the group consisting of carboxyl, aminocarbonyl, alkylsulfonylaminocarbonyl and alkoxycarbonyl;
wherein R¹¹ is selected from the group consisting of haloalkyl, alkyl, aralkyl, cycloalkyl and aryl optionally substituted with one or more radicals selected from alkylthio, nitro and alkylsulfonyl; and
wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or
wherein R¹² together with ring E forms a naphthyl radical; or an isomer or pharmaceutically acceptable salt thereof; and
including the diastereomers, enantiomers, racemates, tautomers, salts, esters, amides and prodrugs thereof.

A related class of compounds useful as Cox-2 selective inhibitors in the present invention is described by Formulas IV and V: 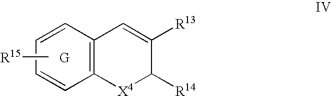
wherein X⁴ is selected from O or S or NR^{a};
wherein R^{a} is alkyl;
wherein R¹³ is selected from carboxyl, aminocarbonyl, alkylsulfonylaminocarbonyl and alkoxycarbonyl;
wherein R¹⁴ is selected from haloalkyl, alkyl, aralkyl, cycloalkyl and aryl optionally substituted with one or more radicals selected from alkylthio, nitro and alkylsulfonyl; and
wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R¹⁵ together with ring G forms a naphthyl radical;
or an isomer or pharmaceutically acceptable salt thereof.

Formula V is: 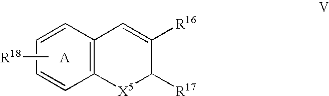
wherein:
X⁵ is selected from the group consisting of O or S or NR^{b};
R^{b} is alkyl;
R¹⁶ is selected from the group consisting of carboxyl, aminocarbonyl, alkylsulfonylaminocarbonyl and alkoxycarbonyl;
R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;
or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of lower haloalkyl, lower cycloalkyl and phenyl; and
R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R¹⁸ together with ring A forms a naphthyl radical;
or an isomer or pharmaceutically acceptable salt thereof.

The Cox-2 selective inhibitor may also be a compound of Formula V, wherein:
X⁵ is selected from the group consisting of oxygen and sulfur;
R¹⁶ is carboxyl;
R¹⁷ is lower haloalkyl; and
R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;
or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or
wherein R² together with ring A forms a naphthyl radical;
or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting trifluoromethyl and pentafluoroethyl; and
R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;
or an isomer or prodrug thereof.

The Cox-2 selective inhibitor of the present invention can also be a compound having the structure of Formula VI: 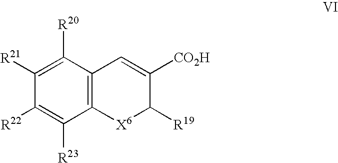
wherein:
X⁶ is selected from the group consisting of O and S;
R¹⁹ is lower haloalkyl;
R²⁰ is selected from the group consisting of hydrido, and halo;
R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R²² is selected from the group consisting of hydrido, lower alkyl, halo, lower alkoxy, and aryl; and
R²³ is selected from the group consisting of the group consisting of hydrido, halo, lower alkyl, lower alkoxy, and aryl;
or an isomer or prodrug thereof.

The Cox-2 selective inhibitor can also be a compound having the structure of Formula VI, wherein:
X⁶ is selected from the group consisting of O and S;
R¹⁹ is selected from the group consisting of trifluoromethyl and pentafluoroethyl;
R²⁰ is selected from the group consisting of hydrido, chloro, and fluoro;
R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R²² is selected from the group consisting of hydrido, methyl, ethyl, isopropyl, tert-butyl, chloro, methoxy, diethylamino, and phenyl; and
R²³ is selected from the group consisting of hydrido, chloro, bromo, fluoro, methyl, ethyl, tert-butyl, methoxy, and phenyl;
or an isomer or prodrug thereof.

The meaning of any substituent at any one occurrence in any general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and diaikoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as: 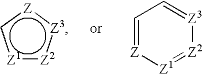
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 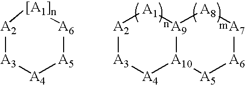
where:
when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m is greater than or equal to 0, atoms separated by 2 atoms (i.e., A₁ and A₄) are sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂(—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 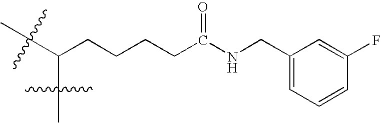
may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

Examples of chromene Cox-2 inhibitors that are suitable for use with the methods and compositions of the present invention include any one or more of: 6-nitro-2-trifluoromethyl-2H-1-benzopyran-3-carboxylic acid; 6-chloro-8-methyl-2-trifluoromethyl-2H-1-benzopyran-3-carboxylic acid; (S)-6-chloro-7-(1,1-dimethylethyl)-2-trifluoromethyl-2H-1-benzopyran-3-carboxylic acid; 2-trifluoromethyl-2H-naphthol[2,3-b]pyran-3-carboxylic acid; 6-chloro-7-(4-nitrophenoxy)-2-(trifluoromethyl)-2H-1-benzopyran-3-carboxylic acid; (S)-6,8-dichloro-2-(trifluoromethyl)-2H-1-benzopyran-3-carboxylic acid; 6-chloro-2-(trifluoromethyl)-4-phenyl-2H-1-benzopyran-3-carboxylic acid; 6-(4-hydroxybenzoyl)-2-(trifluoromethyl)-2H-1-benzopyran-3-carboxylic acid; 2-(trifluoromethyl)-6-[(trifluoromethyl)thiol]-2H-1-benzothiopyran-3-carboxylic acid; 6,8-dichloro-2-trifluoromethyl-2H-1-benzothiopyran-3 carboxylic acid; 6-(1,1-dimethylethyl)-2-(trifluoromethyl)-2H-1-benzothiopyran-3-carboxylic acid; 6,7-difluoro-1,2-dihydro-2-(trifluoromethyl)-3-quinolinecarboxylic acid; 6-chloro-1,2-dihydro-1-methyl-2-(trifluoromethyl)-3-quinolinecarboxylic acid; 6-chloro-2-(trifluoromethyl)-1,2-dihydro[1,8]naphthyridine-3-carboxylic acid; (S)-6-chloro-1,2-dihydro-2-(trifluoromethyl)-3-quinolinecarboxylic acid; (2S)-6,8-dimethyl-2-(trifluoromethyl)-2H-chromene-3-carboxylic acid; (2S)-8-ethyl-6-(trifluoromethoxy)-2-(trifluoromethyl)-2H-chromene-3-carboxylic acid; (2S)-6-chloro-5,7-dimethyl-2-(trifluoromethyl)-2H-chromene-3-carboxylic acid; 6-chloro-2-trifluoromethyl-2H-1-benzopyran-3-carboxylic acid; 6-chloro-7-methyl-2-trifluoromethyl-2H-1-benzopyran-3-carboxylic acid; 8-(1-methylethyl)-2-trifluoromethyl-2H-1-benzopyran-3-carboxylic acid; 6-chloro-7-(1,1-dimethylethyl)-2-trifluoromethyl-2H-1-benzopyran-3-carboxylic acid; 6-chloro-8-(1-methylethyl)-2-trifluoromethyl-2H-1-benzopyran-3-carboxylic acid; 2-trifluoromethyl-3H-naphthopyran-3-carboxylic acid; 7-(1,1-dimethylethyl)-2-trifluoromethyl-2H-1-benzopyran-3-carboxylic acid; 6-bromo-2-trifluoromethyl-2H-1-benzopyran-3-carboxylic acid; 8-chloro-2-trifluoromethyl-2H-1-benzopyran-3-carboxylic acid; 6-trifluoromethoxy-2-trifluoromethyl-2H-1-benzopyran-3-carboxylic acid; 5,7-dichloro-2-trifluoromethyl-2H-1-benzopyran-3-carboxylic acid; 8-phenyl-2-trifluoromethyl-2H-1-benzopyran-3-carboxylic acid; 7,8-dimethyl-2-trifluoromethyl-2H-1-benzopyran-3-carboxylic acid; 6,8-bis(dimethylethyl)-2-trifluoromethyl-2H-1-benzopyran-3-carboxylic acid; 7-(1-methylethyl)-2-trifluoromethyl-2H-1-benzopyran-3-carboxylic acid; 7-phenyl-2-trifluoromethyl-2H-1-benzopyran-3-carboxylic acid; 6-chloro-7-ethyl-2-trifluoromethyl-2H-1-benzopyran-3-carboxylic acid; 6-chloro-8-ethyl-2-trifluoromethyl-2H-1-benzopyran-3-carboxylic acid; 6-chloro-7-phenyl-2-trifluoromethyl-2H-1-benzopyran-3-carboxylic acid; 6,7-dichloro-2-trifluoromethyl-2H-1-benzopyran-3-carboxylic acid; 6,8-dichloro-2-trifluoromethyl-2H-1-benzopyran-3-carboxylic acid; 2-trifluoromethyl-3H-naptho[2,1-b]pyran-3-carboxylic acid; 6-chloro-8-methyl-2-trifluoromethyl-2H-1-benzopyran-3-carboxylic acid; 8-chloro-6-methyl-2-trifluoromethyl-2H-1-benzopyran-3-carboxylic acid; 8-chloro-6-methoxy-2-trifluoromethyl-2H-1-benzopyran-3-carboxylic acid; 6-bromo-8-chloro-2-trifluoromethyl-2H-1-benzopyran-3-carboxylic acid; 8-bromo-6-fluoro-2-trifluoromethyl-2H-1-benzopyran-3-carboxylic acid; 8-bromo-6-methyl-2-trifluoromethyl-2H-1-benzopyran-3-carboxylic acid; 8-bromo-5-fluoro-2-trifluoromethyl-2H-1-benzopyran-3-carboxylic acid; 6-chloro-8-fluoro-2-trifluoromethyl-2H-1-benzopyran-3-carboxylic acid; 6-bromo-8-methoxy-2-trifluoromethyl-2H-1-benzopyran-3-carboxylic acid; 6-[[(phenylmethyl)amino]sulfonyl]-2-trifluoromethyl-2H-1-benzopyran-3-carboxylic acid; 6-[(dimethylamino)sulfonyl]-2-trifluoromethyl-2H-1-benzopyran-3-carboxylic acid; 6-[(methylamino)sulfonyl]-2-trifluoromethyl-2H-1-benzopyran-3-carboxylic acid; 6-[(4-morpholino)sulfonyl]-2-trifluoromethyl-2H-1-benzopyran-3-carboxylic acid; 6-[(1,1-dimethylethyl)aminosulfonyl]-2-trifluoromethyl-2H-1-benzopyran-3-carboxylic acid; 6-[(2-methylpropyl)aminosulfonyl]-2-trifluoromethyl-2H-1-benzopyran-3-carboxylic acid; 6-methylsulfonyl-2-trifluoromethyl-2H-1-benzopyran-3-carboxylic acid; 8-chloro-6-[[(phenylmethyl)amino]sulfonyl]-2-trifluoromethyl-2H-1-benzopyran-3-carboxylic acid; 6-phenylacetyl-2-trifluoromethyl-2H-1-benzopyran-3-carboxylic acid; 6,8-dibromo-2-trifluoromethyl-2H-1-benzopyran-3-carboxylic acid; 8-chloro-5,6-dimethyl-2-trifluoromethyl-2H-1-benzopyran-3-carboxylic acid; 6,8-dichloro-(S)-2-trifluoromethyl-2H-1-benzopyran-3-carboxylic acid; 6-benzylsulfonyl-2-trifluoromethyl-2H-1-benzopyran-3-carboxylic acid; 6-[[N-(2-furylmethyl)amino]sulfonyl]-2-trifluoromethyl-2H-1-benzopyran-3-carboxylic acid; 6-[[N-(2-phenylethyl)amino]sulfonyl]-2-trifluoromethyl-2H-1-benzopyran-3-carboxylic acid; 6-iodo-2-trifluoromethyl-2H-1-benzopyran-3-carboxylic acid; 7-(1,1-dimethylethyl)-2-pentafluoroethyl-2H-1-benzopyran-3-carboxylic acid; 6-chloro-2-trifluoromethyl-2H-1-benzothiopyran-3-carboxylic acid; 6-chloro-2-trifluoromethyl-2H-1-benzopyran-3-carboxylic acid; (S)-6-chloro-2-trifluoromethyl-2H-1-benzopyran-3-carboxylic acid; 6-chloro-7-(1,1-dimethylethyl)-2-trifluoromethyl-2H-1-benzopyran-3-carboxylic acid; (S)-6-chloro-7-(1,1-dimethylethyl)-2-(trifluoromethyl)-2H-1-benzopyran-3-carboxylic acid; 6-trifluoromethoxy-2-trifluoromethyl-2H-1-benzopyran-3-carboxylic acid; (S)-6-trifluoromethoxy-2-trifluoromethyl-2H-1-benzopyran-3-carboxylic acid; 6-formyl-2-(trifluoromethyl)-2H-1-benzopyran-3-carboxylic acid; 6-(difluoromethyl)-2-(trifluoromethyl)-2H-1-benzopyran-3-carboxylic acid; 6,8-dichloro-7-methyl-2-(trifluoromethyl)-2H-1-benzopyran-3-carboxylic acid; 6,8-dichloro-2-trifluoromethyl-2H-1-benzopyran-3-carboxylic acid; (S)-6,8-dichloro-2-(trifluoromethyl)-2H-1-benzopyran-3-carboxylic acid; 6-chloro-1,2-dihydro-2-(trifluoromethyl)-3-quinolinecarboxylic acid; (S)-6-chloro-1,2-dihydro-2-(trifluoromethyl)-3-quinolinecarboxylic acid; 6,8-dichloro-1,2-dihydro-2-(trifluoromethyl)-3-quinolinecarboxylic acid; 7-(1,1-dimethylethyl)-2-trifluoromethyl-2H-1-benzopyran-3-carboxylic acid; 6,7-dichloro-2-trifluoromethyl-2H-1-benzopyran-3-carboxylic acid; 5,6-dichloro-2-(trifluoromethyl)-2H-1-benzopyran-3-carboxylic acid; 2,6-bis(trifluoromethyl)-2H-1-benzopyran-3-carboxylic acid; 5,6,7-trichloro-2-(trifluoromethyl)-2H-1-benzopyran-3-carboxylic acid; 6,7,8-trichloro-2-(trifluoromethyl)-2H-1-benzopyran-3-carboxylic acid; 6-iodo-1,2-dihydro-2-(trifluoromethyl)-3-quinolinecarboxylic acid; 6-bromo-1,2-dihydro-2-(trifluoromethyl)-3-quinolinecarboxylic acid; 6-chloro-7-methyl-2-(trifluoromethyl)-2H-1-benzothiopyran-3-carboxylic acid; 6,8-dichloro-2-trifluoromethyl-2H-1-benzothiopyran-3-carboxylic acid; prodrugs thereof, salts thereof; isomers thereof; and/or mixtures thereof.

Further examples of preferred chromene Cox-2 inhibitors include (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, prodrugs thereof, salts thereof, isomers thereof, and/or mixtures thereof.

In another embodiment of the invention, the Cox-2 inhibitor can be selected from the class of tricyclic Cox-2 selective inhibitors represented by the general structure of formula VII: 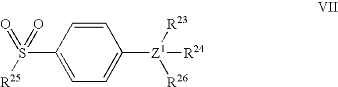
wherein:
Z¹ is selected from the group consisting of partially unsaturated or unsaturated heterocyclyl and partially unsaturated or unsaturated carbocyclic rings;
R²³ is optionally present and if present is selected from an R²⁶ group;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; prodrugs thereof; salts thereof; isomers thereof; and/or mixtures thereof.

In one embodiment of the invention, the tricyclic Cox-2 selective inhibitor comprises at least one compound chosen from celecoxib, parecoxib, deracoxib, valdecoxib, lumiracoxib, etoricoxib, rofecoxib, tilmacoxib, cimicoxib, imrecoxib, prodrugs thereof, salts thereof, isomers thereof, and/or mixtures thereof.

In another embodiment of the invention, the Cox-2 selective inhibitor represented by the above Formula VII is chosen from those compounds, illustrated in Table 1, which includes celecoxib (B-1), valdecoxib (B-2), deracoxib (B-3), rofecoxib (B-4), etoricoxib (MK-663; B-5), tilmacoxib (JTE-522) (B-6), cimicoxib (B-7), prodrugs thereof, salts thereof, isomers thereof, and/or mixtures thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-9 (U.S. Pat. No. 5,840,924); compound B-10 (WO 00/25779); imrecoxib (BAP-909) (4-(4-methane-sulfonyl-phenyl-)-1-propyl-3-p-tolyl-1,5-dihydropyrrol-2-one); cimicoxib (4-[4-chloro-5-(3-fluoro-4-methoxyphenyl)-1H-imidzol-1-yl]benzenesulfonamide—CAS RN 265114-23-6); tilmacoxib (4-(4-cyclohexyl-2-methyloxazol-5-yl)-2-fluorobenzenesulfonamide—JTE-522, CAS 180200-68-4); and etoricoxib (CAS RN 202409-33-4, MK-663, SC-86218, and in WO 98/03484).

TABLE 1
Examples of Tricyclic Cox-2 Selective Inhibitors

| Compound | Common | Chemical |
|---|---|---|
| No. | name | name |
| B-1 | celecoxib | 4-[5-(4-methylphenyl)-3-(trifluoromethyl)-1H- |
| | | pyrazol-1-yl] benzenesulfonamide |
| B-2 | valdecoxib | 4-(5-methyl-3-phenyl-4-isoxazolyl) |
| | | benzenesulfonamide |
| B-3 | deracoxib | 4-[3-(difluoromethyl)-5-(3-fluoro-4- |
| | | methoxyphenyl)-1H-pyrazol-1- |
| | | yl]benzenesulfonamide |
| B-4 | rofecoxib | 4-[4-(methylsulfonyl)phenyl]-3-phenyl-2(5H)- |
| | | furanone |
| B-5 | etoricoxib | 2,3′-bipyridine, 5-chloro-6′-methyl-3-[4- |
| | | [methylsulfonyl]phenyl]-; or [2] 5-chloro-6′- |
| | | methyl-3-[p-[methylsulfonyl]phenyl]-2,3′- |
| | | bipyridine |
| B-6 | tilmacoxib | 4-(4-cyclohexyl-2-methyloxazol-5-yl)-2- |
| | | fluorobenzenesulfonamide |
| B-7 | cimicoxib | 4-[4-chloro-5-(3-fluoro-4-methoxyphenyl)-1H- |
| | | imidzol-1-yl]benzenesulfonamide |
| B-8 | parecoxib | N-[[p-(5-methyl-3-phenyl-4-isox- |
| | | azolyl)phenyl]sulfonyl]propionamide |

In yet another embodiment of the invention, the Cox-2 selective inhibitor is selected from the group consisting of celecoxib, valdecoxib, rofecoxib, etoricoxib, tilmacoxib, cimicoxib, deracoxib, parecoxib, prodrugs thereof, salts thereof, isomers thereof, and/or mixtures thereof. Even more preferred still is that the Cox-2 selective inhibitor is celecoxib.

In another embodiment, the tricyclic Cox-2 selective inhibitor, parecoxib (B-8), N-[[4-(5-methyl-3-phenyl-4-isoxazo-lyl)phenyl]sulfonyl]-; or (2) N-[[p-(5-methyl-3-phenyl-4-isox-azolyl)phenyl]sulfonyl]propionamide; CAS No. 198470-84-7 (See, U.S. Pat. No. 5,932,598), which is a therapeutically effective prodrug of the tricyclic Cox-2 selective inhibitor valdecoxib, compound B-2, (See, U.S. Pat. No. 5,633,272), may be advantageously employed as the Cox-2 inhibitor of the present invention.

A preferred form of parecoxib is sodium parecoxib, which is available as Dynastat®.

Another tricyclic Cox-2 selective inhibitor useful in the present invention is the compound ABT-963, having the structure: 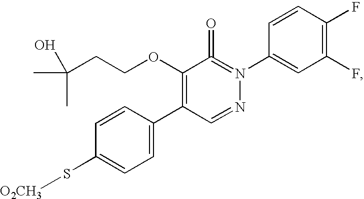
which has been previously described in International Publication Number WO 00/24719.

In a further embodiment of the invention, the Cox-2 inhibitor can be selected from the class of phenylacetic acid derivative Cox-2 selective inhibitors represented by the general structure of formula VIII: 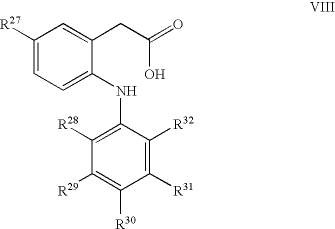
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxyl;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula VIII,

wherein:

R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula VIII,

wherein:

R²⁷ is propyl;
R²⁸ and R³⁰ are chloro;
R²⁹ and R³¹ are methyl; and
R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula VIII,

wherein:

R²⁷ is methyl;
R²⁸ is fluoro;
R³² is chloro; and
R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

In certain aspects of the present invention, the Cox-2 selective inhibitor may be a Cox-2 selective inhibitor that is other than any tricyclic Cox-2 selective inhibitor described by formula VII. For example, the Cox-2 selective inhibitor may be a chromene Cox-2 inhibitor, which is a class of Cox-2 selective inhibitor that is other than a tricylic Cox-2 selective inhibitor. Likewise, the Cox-2 selective inhibitor may be any compound described by formula VIII, such as lumiracoxib, which is other than a tricyclic Cox-2 selective inhibitor. Thus, in some embodiments, the present invention encompasses any Cox-2 selective inhibitor that is other than a tricyclic Cox-2 selective inhibitor that is described by formula VII, whether such a Cox-2 selective inhibitor is now known or later developed.

In other aspects of the present invention, the Cox-2 selective inhibitor may be at least one compound or class of compounds chosen from Table 2, isomers thereof, salts thereof, and/or mixtures thereof. However, the present invention should not be construed as being limited to any particular one of the Cox-2 selective inhibitors described herein. Indeed, it should be understood that the present invention encompasses any compound that can be shown to act as an inhibitor of the Cox-2 enzyme, whether such a compound is now known, later developed, or even later recognized as having Cox-2 inhibitory activity.

TABLE 2
Additional Cox-2 Selective Inhibitors

| | | Trade | | | | |
|---|---|---|---|---|---|---|
| No. | Generic Name/Compound Name | Name(s) | Drug Class/Mode of Action | Dose | Manufacturer | Reference |
| B11 | Nimesulide | | | | | |
| B12 | Flosulide | | | | | |
| B13 | NS-398 | | | | | CAS RN 123653-11-2 |
| | N-(2-cyclohexyloxynitrophenyl) | | | | | Yoshimi, N. et al., in |
| | methane sulfonamide | | | | | Japanese J. Cancer |
| | | | | | | Res., 90(4): 406-412 |
| | | | | | | (1999) |
| B14 | L-745337 | | | | | |
| B15 | RWJ-63556 | | | | | Kirchner et al., in J |
| | | | | | | Pharmacol Exp Ther |
| | | | | | | 282, 1094-1101 |
| | | | | | | (1997) |
| B16 | L-784512 | | | | | |
| B17 | N-(2- | | diarylmethylidenefuran | | | U.S. Pat. No. |
| | cyclohexyloxynitrophenyl)methane | | derivatives | | | 6,180,651 |
| | sulfonamide, and (E)-4-[(4- | | | | | |
| | methylphenyl)(tetrahydro-2-oxo-3- | | | | | |
| | furanylidene) | | | | | |
| | methyl]benzenesulfonamide | | | | | |
| B18 | Darbufelone | | | | Pfizer | |
| B19 | CS-502 | | | | Sankyo | |
| B20 | LAS 34475 | | | | Almirall | |
| | | | | | Profesfarma | |
| B21 | LAS 34555 | | | | Almirall | |
| | | | | | Profesfarma | |
| B22 | S-33516 | | | | Servier | |
| B23 | SD 8381 | | | | Pharmacia | U.S. Pat. No. |
| | | | | | | 6,034,256 |
| B24 | BMS-347070 | | | | Bristol Myers | U.S. Pat. No. |
| | | | | | Squibb | 6,180,651 |
| B25 | MK-966 | | | | Merck | |
| B26 | L-783003 | | | | Merck | |
| B27 | T-614 | | | | Toyama | |
| B28 | D-1367 | | | | Chiroscience | |
| B29 | L-748731 | | | | Merck | |
| B30 | CT3 | | | | Atlantic | |
| | | | | | Pharmaceutical | |
| B31 | CGP-28238 | | | | Novartis | |
| B32 | BF-389 | | | | Biofor/Scherer | |
| B33 | GR-253035 | | | | Glaxo Wellcome | |
| B34 | 6-dioxo-9H-purin-8-yl-cinnamic acid | | | | Glaxo Wellcome | |
| B35 | S-2474 | | | | Shionogi | |
| B36 | | | Multibinding compounds | | | U.S. Pat. No. |
| | | | containing from 2 to 10 | | | 6,395,724 |
| | | | ligands covanlently attached | | | |
| | | | to one or more linkers | | | |
| B37 | | | Conjugated linoleic acid | | | U.S. Pat. No. |
| | | | derivatives | | | 6,077,868 |
| B38 | | | Heterocyclic aromatic oxazole | | | U.S. Pat. Nos. |
| | | | compounds | | | 5,994,381 and |
| | | | | | | 6,362,209 |
| B39 | | | Miscellaneous compounds | | | U.S. Pat. Nos. |
| | | | | | | 6,596,736, |
| | | | | | | 6,369,275, |
| | | | | | | 6,127,545, |
| | | | | | | 6,130,334, |
| | | | | | | 6,204,387, |
| | | | | | | 6,071,936, 6,001,843 |
| | | | | | | and 6,040,450 |
| B40 | | | Diarylbenzopyran derivatives | | | U.S. Pat. No. |
| | | | | | | 6,340,694 |
| B41 | | | 1-(4-sulfamylaryl)-3- | | | U.S. Pat. No. |
| | | | substituted-5-aryl-2- | | | 6,376,519 |
| | | | pyrazolines | | | |
| B42 | | | Heterocycle compounds | | | U.S. Pat. No. |
| | | | | | | 6,153,787 |
| B43 | | | 2,3,5-trisubstituted pyridines | | | U.S. Pat. No. |
| | | | | | | 6,046,217 |
| B44 | | | Diaryl bicyclic heterocycles | | | U.S. Pat. No. |
| | | | | | | 6,329,421 |
| B45 | | | Salts of 5-amino or | | | U.S. Pat. No. |
| | | | substituted amino 1,2,3- | | | 6,239,137 |
| | | | triazole compounds | | | |
| B46 | | | Pyrazole derivatives | | | U.S. Pat. No. |
| | | | | | | 6,136,831 |
| B47 | | | Substituted derivatives of | | | U.S. Pat. No. |
| | | | benzosulphonamides | | | 6,297,282 |
| B48 | 3-phenyl-4-(4- | | Phenyl heterocycles | | | U.S. Pat. Nos. |
| | (methylsulfonyl)phenyl)-2-(2H)- | | | | | 5,474,995 and |
| | furanone | | | | | 6,239,173 |
| B49 | | | Bicycliccarbonyl indole | | | U.S. Pat. No. |
| | | | compounds | | | 6,303,628 |
| B50 | | | Benzimidazole compounds | | | U.S. Pat. No. |
| | | | | | | 6,310,079 |
| B51 | | | Indole compounds | | | U.S. Pat. No. |
| | | | | | | 6,300,363 |
| B52 | | | Aryl phenylhydrazides | | | U.S. Pat. No. |
| | | | | | | 6,077,869 |
| B53 | | | 2-aryloxy, 4-aryl furan-2-ones | | | U.S. Pat. No. |
| | | | | | | 6,140,515 |
| B54 | | | Bisaryl compounds | | | U.S. Pat. No. |
| | | | | | | 5,994,379 |
| B55 | | | 1,5-diarylpyrazoles | | | U.S. Pat. No. |
| | | | | | | 6,028,202 |
| B56 | | | 2-substituted imidazoles | | | U.S. Pat. No. |
| | | | | | | 6,040,320 |
| B57 | | | 1,3- and 2,3-diarylcycloalkano | | | U.S. Pat. No. |
| | | | and cycloalkeno pyrazoles | | | 6,083,969 |
| B58 | | | Esters derived from | | | U.S. Pat. No. |
| | | | indolealkanols and novel | | | 6,306,890 |
| | | | amides derived from | | | |
| | | | indolealkylamides | | | |
| B59 | | | Pyridazinone compounds | | | U.S. Pat. No. |
| | | | | | | 6,307,047 |
| B60 | | | Benzosulphonamide | | | U.S. Pat. No. |
| | | | derivatives | | | 6,004,948 |
| B61 | | | Methanesulfonyl-biphenyl | | | U.S. Pat. No. |
| | | | derivatives | | | 6,583,321 |
| B62 | | | 1H-indole derivatives | | | U.S. Pat. No. |
| | | | | | | 6,599,929 |
| B63 | N-(2-hydroxyethyl)-4-[5-(4- | | Certain prodrugs of Cox-2 | | | U.S. Pat. Nos. |
| | methylphenyl)-3-(trifluoromethyl)- | | inhibitors | | | 6,436,967 and |
| | 1H-pyrazol-1- | | | | | 6,613,790 |
| | yl]benzenesulfonamide, N,N-bis(2- | | | | | |
| | hydroxyethyl)-4-[5-(4-methylphenyl)- | | | | | |
| | 3-(trifluoromethyl)-1H-pyrazol-1- | | | | | |
| | yl]benzenesulfonamide | | | | | |
| B64 | | | Sulfamoylheteroaryl pyrazole | | | U.S. Pat. No. |
| | | | compounds | | | 6,583,321 |
| B65 | | | Heteroaryl substituted | | | U.S. Pat. No. |
| | | | amidinyl and imidazolyl | | | 6,555,563 |
| | | | compounds | | | |
| B66 | | | Substituted hydroxamic acid | | | U.S. Pat. Nos. |
| | | | derivatives | | | 6,432,999, |
| | | | | | | 6,512,121, and |
| | | | | | | 6,515,014 |
| B67 | | | Pyrazolopyridine compounds | | | U.S. Pat. No. |
| | | | | | | 6,498,166 |
| B68 | | | 4,5-diaryl-3(2H)-furanone | | | U.S. Pat. No. |
| | | | derivatives | | | 6,492,416 |
| B69 | | | 2-phenyl-1,2- | | | U.S. Pat. No. |
| | | | benzisoselenazol-3(2H)-one | | | 6,492,416 |
| | | | derivatives and 2- | | | |
| | | | phenylcarbomyl- | | | |
| | | | phenylselenyl derivatives | | | |
| B70 | | | Pyrones | | | U.S. Pat. No. |
| | | | | | | 6,465,509 |
| B71 | | | Organically synthesized or | | | U.S. Published |
| | | | purified from plant sources, | | | Application No. |
| | | | free-B-ring flavanoids | | | 2003/0165588 |
| B72 | | | Heterocyclo-alkylsulfonyl | | | European Patent |
| | | | pyrazoles | | | Application No. EP |
| | | | | | | 1312367 |
| B73 | | | 2-phenylpyran-4-one | | | U.S. Pat. No. |
| | | | derivatives | | | 6,518,303 |
| B74 | | | Sulfonylphenylpyrazoles | | | U.S. Pat. No. |
| | | | | | | 6,472,416 |
| B75 | | | 2,3-diaryl-pyrazolo[1,5- | | | U.S. Pat. No. |
| | | | b]pyridazines | | | 6,451,794 |
| B76 | | | (Methylsulfonyl)phenyl | | | U.S. Pat. Nos. |
| | | | furanones | | | 6,169,188, |
| | | | | | | 6,020,343, and |
| | | | | | | 5,981,576 |
| B77 | | | Diaryl-2-(5H)-furanones | | | U.S. Pat. No. |
| | | | | | | 6,222,048 |
| B78 | | | 3,4-diaryl-2-hydroxy-2,5- | | | U.S. Pat. No. |
| | | | dihydrofurans | | | 6,057,319 |
| B79 | | | Carbocyclic sulfonamides | | | U.S. Pat. No. |
| | | | | | | 6,046,236 |
| B80 | | | Oxazole derivatives | | | U.S Pat. Nos. |
| | | | | | | 6,002,014 and |
| | | | | | | 5,945,539 |
| B81 | | | C-nitroso compounds | | | U.S. Pat. Nos. |
| | | | | | | 6,359,182 and |
| | | | | | | 6,538,116 |
| B82 | | | Substituted pyridines | | | U.S. Published |
| | | | | | | Application No. |
| | | | | | | 2003/0065011 |
| B83 | | | Substituted indole derivatives | | | U.S. Published |
| | | | | | | Application No. |
| | | | | | | 2003/0207897 |
| B84 | meloxicam | | | | | CAS registry number |
| | | | | | | 71125-38-7 |
| B85 | RS 57067 | | | | | CAS registry number |
| | 6-[[5-(4-chlorobenzoyl)-1,4-dimethyl- | | | | | 179382-91-3 |
| | 1H-pyrrol-2-yl]methyl]-3(2H)- | | | | | |
| | pyridazinone | | | | | |
| B86 | PMI-001 | | Tricyclic Cox-2 inhibitor- | | Phytomedics | |
| | | | botanical extract | | | |
| B87 | 644784 | | Tricyclic Cox-2 inhibitor | | GlaxoSmithKline | |
| B88 | CS-706 (R-109339) | | Tricyclic Cox-2 inhibitor | | Sankyo | |
| B89 | PAC-10549, PAC-10649 | | Non-tricyclic Cox-2 inhibitor | | Pacific, Corp. | |

Examples of specific compounds that are useful as Cox-2 selective inhibitors include, without limitation: 8-acetyl-3-(4-fluorophenyl)-2-(4-methylsulfonyl)phenyl-imidazo(1,2-a)pyridine; 5,5-dimethyl-4-(4-methylsulfonyl)phenyl-3-phenyl-2-(5H)-furanone; 5-(4-fluorophenyl)-1-[4-(methylsulfonyl)phenyl]-3-(trifluoromethyl)pyrazole; 4-(4-fluorophenyl)-5-[4-(methylsulfonyl)phenyl]-1-phenyl-3-(trifluoromethyl)pyrazole; 4-(5-(4-chlorophenyl)-3-(4-methoxyphenyl)-1H-pyrazol-1-yl)benzenesulfonamide; 4-(3,5-bis(4-methylphenyl)-1H-pyrazol-1-yl)benzenesulfonamide; 4-(5-(4-chlorophenyl)-3-phenyl-1H-pyrazol-1-yl)benzenesulfonamide; 4-(3,5-bis(4-methoxyphenyl)-1H-pyrazol-1-yl)benzenesulfonamide; 4-(5-(4-chlorophenyl)-3-(4-methylphenyl)-1H-pyrazol-1-yl)benzenesulfonamide; 4-(5-(4-chlorophenyl)-3-(4-nitrophenyl)-1H-pyrazol-1-yl)benzenesulfonamide; 4-(5-(4-chlorophenyl)-3-(5-chloro-2-thienyl)-1H-pyrazol-1-yl)benzenesulfonamide; 4-(4-chloro-3,5-diphenyl-1H-pyrazol-1-yl)benzenesulfonamide; 4-[5-(4-chlorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[4-chloro-5-(4-chlorophenyl)-3-(trifluoromethyl)-1H-pyrazol-1-yl]benzenesulfonamide; 4-[3-(difluoromethyl)-5-(4-methylphenyl)-1H-pyrazol-1-yl]benzenesulfonamide; 4-[3-(difluoromethyl)-5-phenyl-1H-pyrazol-1-yl]benzenesulfonamide; 4-[3-(difluoromethyl)-5-(4-methoxyphenyl)-1H-pyrazol-1-yl]benzenesulfonamide; 4-[3-cyano-5-(4-fluorophenyl)-1H-pyrazol-1-yl]benzenesulfonamide; 4-[3-(difluoromethyl)-5-(3-fluoro-4-methoxyphenyl)-1H-pyrazol-1-yl]benzenesulfonamide; 4-[5-(3-fluoro-4-methoxyphenyl)-3-(trifluoromethyl)-1H-pyrazol-1-yl]benzenesulfonamide; 4-[4-chloro-5-phenyl-1H-pyrazol-1-yl]benzenesulfonamide; 4-[5-(4-chlorophenyl)-3-(hydroxymethyl)-1H-pyrazol-1-yl]benzenesulfonamide; 4-[5-(4-(N,N-dimethylamino)phenyl)-3-(trifluoromethyl)-1H-pyrazol-1-yl]benzenesulfonamide; 5-(4-fluorophenyl)-6-[4-(methylsulfonyl)phenyl]spiro[2.4]hept-5-ene; 4-[6-(4-fluorophenyl)spiro[2.4]hept-5-en-5-yl]benzenesulfonamide; 6-(4-fluorophenyl)-7-[4-(methylsulfonyl)phenyl]spiro[3.4]oct-6-ene; 5-(3-chloro-4-methoxyphenyl)-6-[4-(methylsulfonyl)phenyl]spiro[2.4]hept-5-ene; 4-[6-(3-chloro-4-methoxyphenyl)spiro[2.4]hept-5-en-5-yl]benzenesulfonamide; 5-(3,5-dichloro-4-methoxyphenyl)-6-[4-(methylsulfonyl)phenyl]spiro[2.4]hept-5-ene; 5-(3-chloro-4-fluorophenyl)-6-[4-(methylsulfonyl)phenyl]spiro[2.4]hept-5-ene; 4-[6-(3,4-dichlorophenyl)spiro[2.4]hept-5-en-5-yl]benzenesulfonamide; 2-(3-chloro-4-fluorophenyl)-4-(4-fluorophenyl)-5-(4-methylsulfonylphenyl)thiazole; 2-(2-chlorophenyl)-4-(4-fluorophenyl)-5-(4-methylsulfonylphenyl)thiazole; 5-(4-fluorophenyl)-4-(4-methylsulfonylphenyl)-2-methylthiazole; 4-(4-fluorophenyl)-5-(4-methylsulfonylphenyl)-2-trifluoromethylthiazole; 4-(4-fluorophenyl)-5-(4-methylsulfonylphenyl)-2-(2-thienyl)thiazole; 4-(4-fluorophenyl)-5-(4-methylsulfonylphenyl)-2-benzylaminothiazole; 4-(4-fluorophenyl)-5-(4-methylsulfonylphenyl)-2-(1-propylamino)thiazole; 2-[(3,5-dichlorophenoxy)methyl)-4-(4-fluorophenyl)-5-[4-(methylsulfonyl)phenyl]thiazole; 5-(4-fluorophenyl)-4-(4-methylsulfonylphenyl)-2-trifluoromethylthiazole; 1-methylsulfonyl-4-[1,1-dimethyl-4-(4-fluorophenyl)cyclopenta-2,4-dien-3-yl]benzene; 4-[4-(4-fluorophenyl)-1,1-dimethylcyclopenta-2,4-dien-3-yl]benzenesulfonamide; 5-(4-fluorophenyl)-6-[4-(methylsulfonyl)phenyl]spiro[2.4]hepta-4,6-diene; 4-[6-(4-fluorophenyl)spiro[2.4]hepta-4,6-dien-5-yl]benzenesulfonamide; 6-(4-fluorophenyl)-2-methoxy-5-[4-(methylsulfonyl)phenyl]-pyridine-3-carbonitrile; 2-bromo-6-(4-fluorophenyl)-5-[4-(methylsulfonyl)phenyl]-pyridine-3-carbonitrile; 6-(4-fluorophenyl)-5-[4-(methylsulfonyl)phenyl]-2-phenyl-pyridine-3-carbonitrile; 4-[2-(4-methylpyridin-2-yl)-4-(trifluoromethyl)-1H-imidazol-1-yl]benzenesulfonamide; 4-[2-(5-methylpyridin-3-yl)-4-(trifluoromethyl)-1H-imidazol-1-yl]benzenesulfonamide; 4-[2-(2-methylpyridin-3-yl)-4-(trifluoromethyl)-1H-imidazol-1-yl]benzenesulfonamide; 3-[1-[4-(methylsulfonyl)phenyl]-4-(trifluoromethyl)-1H-imidazol-2-yl]pyridine; 2-[1-[4-(methylsulfonyl)phenyl-4-(trifluoromethyl)-1H-imidazol-2-yl]pyridine; 2-methyl-4-[1-[4-(methylsulfonyl)phenyl-4-(trifluoromethyl)-1H-imidazol-2-yl]pyridine; 2-methyl-6-[1-[4-(methylsulfonyl)phenyl-4-(trifluoromethyl)-1H-imidazol-2-yl]pyridine; 4-[2-(6-methylpyridin-3-yl)-4-(trifluoromethyl)-1H-imidazol-1-yl]benzenesulfonamide; 2-(3,4-difluorophenyl)-1-[4-(methylsulfonyl)phenyl]-4-(trifluoromethyl)-1H-imidazole; 4-[2-(4-methylphenyl)-4-(trifluoromethyl)-1H-imidazol-1-yl]benzenesulfonamide; 2-(4-chlorophenyl)-1-[4-(methylsulfonyl)phenyl]-4-methyl-1H-imidazole; 2-(4-chlorophenyl)-1-[4-(methylsulfonyl)phenyl]-4-phenyl-1H-imidazole; 2-(4-chlorophenyl)-4-(4-fluorophenyl)-1-[4-(methylsulfonyl)phenyl]-1H-imidazole; 2-(3-fluoro-4-methoxyphenyl)-1-[4-(methylsulfonyl)phenyl-4-(trifluoromethyl)-1H-imidazole; 1-[4-(methylsulfonyl)phenyl]-2-phenyl-4-trifluoromethyl-1H-imidazole; 2-(4-methylphenyl)-1-[4-(methylsulfonyl)phenyl]-4-trifluoromethyl-1H-imidazole; 4-[2-(3-chloro-4-methylphenyl)-4-(trifluoromethyl)-1H-imidazol-1-yl]benzenesulfonamide; 2-(3-fluoro-5-methylphenyl)-1-[4-(methylsulfonyl)phenyl]-4-(trifluoromethyl)-1H-imidazole; 4-[2-(3-fluoro-5-methylphenyl)-4-(trifluoromethyl)-1H-imidazol-1-yl]benzenesulfonamide; 2-(3-methylphenyl)-1-[4-(methylsulfonyl)phenyl]-4-trifluoromethyl-1H-imidazole; 4-[2-(3-methylphenyl)-4-trifluoromethyl-1H-imidazol-1-yl]benzenesulfonamide; 1-[4-(methylsulfonyl)phenyl]-2-(3-chlorophenyl)-4-trifluoromethyl-1H-imidazole; 4-[2-(3-chlorophenyl)-4-trifluoromethyl-1H-imidazol-1-yl]benzenesulfonamide; 4-[2-phenyl-4-trifluoromethyl-1H-imidazol-1-yl]benzenesulfonamide; 4-[2-(4-methoxy-3-chlorophenyl)-4-trifluoromethyl-1H-imidazol-1-yl]benzenesulfonamide; 1-allyl-4-(4-fluorophenyl)-3-[4-(methylsulfonyl)phenyl]-5-(trifluoromethyl)-1H-pyrazole; 4-[1-ethyl-4-(4-fluorophenyl)-5-(trifluoromethyl)-1H-pyrazol-3-yl]benzenesulfonamide; N-phenyl-[4-(4-luorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate; 4-(4-fluorophenyl)-3-[4-(methylsulfonyl)phenyl]-1-(2-phenylethyl)-1H-pyrazole; 4-(4-fluorophenyl)-3-[4-(methylsulfonyl)phenyl]-1-(2-phenylethyl)-5-(trifluoromethyl)pyrazole; 1-ethyl-4-(4-fluorophenyl)-3-[4-(methylsulfonyl)phenyl]-5-(trifluoromethyl)-1H-pyrazole; 5-(4-fluorophenyl)-4-(4-methylsulfonylphenyl)-2-trifluoromethyl-1H-imidazole; 4-[4-(methylsulfonyl)phenyl]-5-(2-thiophenyl)-2-(trifluoromethyl)-1H-imidazole; 5-(4-fluorophenyl)-2-methoxy-4-[4-(methylsulfonyl)phenyl]-6-(trifluoromethyl)pyridine; 2-ethoxy-5-(4-fluorophenyl)-4-[4-(methylsulfonyl)phenyl]-6-(trifluoromethyl)pyridine; 5-(4-fluorophenyl)-4-[4-(methylsulfonyl)phenyl]-2-(2-propynyloxy)-6-(trifluoromethyl)pyridine; 2-bromo-5-(4-fluorophenyl)-4-[4-(methylsulfonyl)phenyl]-6-(trifluoromethyl)pyridine; 4-[2-(3-chloro-4-methoxyphenyl)-4,5-difluorophenyl]benzenesulfonamide; 1-(4-fluorophenyl)-2-[4-(methylsulfonyl)phenyl]benzene; 5-difluoromethyl-4-(4-methylsulfonylphenyl)-3-phenylisoxazole; 4-[3-ethyl-5-phenylisoxazol-4-yl]benzenesulfonamide; 4-[5-difluoromethyl-3-phenylisoxazol-4-yl]benzenesulfonamide; 4-[5-hydroxymethyl-3-phenylisoxazol-4-yl]benzenesulfonamide; 4-[5-methyl-3-phenyl-isoxazol-4-yl]benzenesulfonamide; 1-[2-(4-fluorophenyl)cyclopenten-1-yl]-4-(methylsulfonyl)benzene; 1-[2-(4-fluoro-2-methylphenyl)cyclopenten-1-y]-4-(methylsulfonyl)benzene; 1-[2-(4-chlorophenyl)cyclopenten-1-yl]-4-(methylsulfonyl)benzene; 1-[2-(2,4-dichlorophenyl)cyclopenten-1-yl]-4-(methylsulfonyl)benzene; 1-[2-(4-trifluoromethylphenyl)cyclopenten-1-yl]-4-(methylsulfonyl)benzene; 1-[2-(4-methylthiophenyl)cyclopenten-1-yl]-4-(methylsulfonyl)benzene; 1-[2-(4-fluorophenyl)-4,4-dimethylcyclopenten-1-yl]-4-(methylsulfonyl)benzene; 4-[2-(4-fluorophenyl)-4,4-dimethylcyclopenten-1-yl]benzenesulfonamide; 1-[2-(4-chlorophenyl)-4,4-dimethylcyclopenten-1-yl]-4-(methylsulfonyl)benzene; 4-[2-(4-chlorophenyl)-4,4-dimethylcyclopenten-1-yl]benzenesulfonamide; 4-[2-(4-fluorophenyl)cyclopenten-1-yl]benzenesulfonamide; 4-[2-(4-chlorophenyl)cyclopenten-1-yl]benzenesulfonamide; 1-[2-(4-methoxyphenyl)cyclopenten-1-yl]-4-(methylsulfonyl)benzene; 1-[2-(2,3-difluorophenyl)cyclopenten-1-yl]-4-(methylsulfonyl)benzene; 4-[2-(3-fluoro-4-methoxyphenyl)cyclopenten-1-yl]benzenesulfonamide; 1-[2-(3-chloro-4-methoxyphenyl)cyclopenten-1-yl]-4-(methylsulfonyl)benzene; 4-[2-(3-chloro-4-fluorophenyl)cyclopenten-1-yl]benzenesulfonamide; 4-[2-(2-methylpyridin-5-yl)cyclopenten-1-yl]benzenesulfonamide; ethyl 2-[4-(4-fluorophenyl)-5-[4-(methylsulfonyl)phenyl]oxazol-2-yl]-2-benzyl-acetate; 2-[4-(4-fluorophenyl)-5-[4-(methylsulfonyl)phenyl]oxazol-2-yl]acetic acid; 2-(tert-butyl)-4-(4-fluorophenyl)-5-[4-(methylsulfonyl)phenyl]oxazole; 4-(4-fluorophenyl)-5-[4-(methylsulfonyl)phenyl]-2-phenyloxazole; 4-(4-fluorophenyl)-2-methyl-5-[4-(methylsulfonyl)phenyl]oxazole; 4-[5-(3-fluoro-4-methoxyphenyl)-2-trifluoromethyl-4-oxazolyl]benzenesulfonamide; salts thereof, isomers thereof, and/or mixtures thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can be synthesized, for example, according to the description in Example 1. Several Cox-2 inhibitors that are suitable for use with the compositions and methods of the present invention may be synthesized by the methods described in, for example, in U.S. Pat. No. 5,466,823 to Talley, et al.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385. Thiophene analogs useful in the present invention can be prepared by methods described in WO 95/00501. Preparation of thiophene analogs is also described in WO 94/15932. Oxazoles useful in the present invention can be prepared by the methods described in WO 95/00501. Preparation of oxazoles is also described in WO 94/27980. Isoxazoles useful in the present invention can be prepared by the methods described in WO 96/25405. Imidazoles useful in the present invention can be prepared by the methods described in WO 96/03388. Preparation of imidazoles is also described in WO 96/03387. Cyclopentene Cox-2 inhibitors useful in the present invention can be prepared by the methods described in U.S. Pat. No. 5,344,991. Preparation of cyclopentene Cox-2 inhibitors is also described in WO 95/00501. Terphenyl compounds useful in the present invention can be prepared by the methods described in WO 96/16934. Thiazole compounds useful in the present invention can be prepared by the methods described in WO 96/03,392. Pyridine compounds useful in the present invention can be prepared by the methods described in WO 96/03392. The preparation of pyridine compounds is also described in WO 96/24,585. Benzopyranopyrazolyl compounds useful in the present invention can be prepared by the methods described in WO 96/09304. Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256. Arylpyridazinones useful in the present invention can be prepared by the methods described in WO 00/24719. Preparation of arylpyridazinones is also described in WO 99/10332. Arylpyridazinones can further be prepared by the methods described in WO 99/10331. 5-Alkyl-2-arylaminophenylacetic acids and derivatives useful in the present invention can be prepared by the methods described in WO 99/11605. Diarylmethylidenefuran derivative Cox-2 selective inhibitors useful in the present invention can be prepared by the methods described in U.S. Pat. No. 6,180,651. The celecoxib used in the compositions and methods of the present invention can be prepared in the manner set forth in U.S. Pat. No. 5,466,823. The valdecoxib used in the compositions and methods of the present invention can be prepared in the manner set forth in U.S. Pat. No. 5,633,272. The parecoxib used in the compositions and methods of the present invention can be prepared in the manner set forth in U.S. Pat. No. 5,932,598. The rofecoxib used in the compositions and methods of the present invention can be prepared in the manner set forth in U.S. Pat. No. 5,474,995. The deracoxib used in the compositions and methods of the present invention can be prepared in the manner set forth in U.S. Pat. No. 5,521,207. The etoricoxib used in the compositions and methods of the present invention can be prepared in the manner set forth in WO 98/03484. The cimicoxib used in the compositions and methods of the present invention can be prepared in the manner set forth in Drugs of the Future, 29(4):325-330 (2004). The imrecoxib used in the compositions and methods of the present invention can be prepared in the manner set forth in Acta Pharmacol Sin, 25(7):927-931 (2004) and patent application No. 00105899.1). The meloxicam used in the compositions and methods of the present invention can be prepared in the manner set forth in U.S. Pat. No. 4,233,299. The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381. The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719. The compound 2-(3,5-difluorophenyl)-3-[4-(methylsulfonyl)phenyl]-2-cyclopenten-1-one used in the compositions and methods of the present invention can be prepared in the manner set forth in EP 863134. The compound 2-[(2-chloro-6-fluorophenyl)amino]-5-methyl-benzeneacetic acid used in the compositions and methods of the present invention can be prepared in the manner set forth in WO 99/11605. The compound N-[2-(cyclohexyloxy)-4-nitrophenyl]methanesulfonamide used in the compositions and methods of the present invention can be prepared in the manner set forth in U.S. Pat. No. 4,885,367. The compound (3Z)-3-[(4-chlorophenyl)[4-(methylsulfonyl) phenyl]methylene]dihydro-2(3H)-furanone used in the compositions and methods of the present invention can be prepared in the manner set forth in U.S. Pat. No. 6,180,651.

Cox-2 inhibitors can also be isolated and purified from natural sources. Cox-2 inhibitors should be of a quality and purity that is conventional in the trade for use in pharmaceutical products.

Another component of the present invention is a nitric oxide- or NO-donating agent. The nitric oxide- or NO-donating agent of the present invention is any compound that donates, releases, or directly or indirectly transfers a nitrogen monoxide species; and/or stimulates the endogenous production of nitric oxide or endothelium-derived relaxing factor (EDRF) in vivo; and/or elevates endogenous levels of nitric oxide or EDRF in vivo. NO-donating agents also include compounds that are substrates for nitric oxide synthase.

Examples of NO-donating agents that are useful in the present invention are presented in Table 3.

TABLE 3
Nitric Oxide-Donating Agents

| Drug Name | Reference |
|---|---|
| Pirsidomine: | de Mey, C., et al., Differential effects of the novel NO-donating |
| N-p-Anisoyl-3-(cis-2,6- | drug pirsidomine and isosorbide dinitrate on the venous |
| dimethylpiperidino)sydnone imine | vascular bed. Eur J Clin Pharmacol. 46: 295-9 (1994). |
| Sodium nitroprusside: | Kowaluk, E. A., et al., Metabolic activation of sodium |
| Disodium pentacyanonitrosyl | nitroprusside to nitric oxide in vascular smooth muscle. J |
| freeate(2-) dihydrate | Pharmacol Exp Ther. 262: 916-22 (1992). |
| Isosorbide dinitrate: | Fujii, S., et al., In vivo three-dimensional EPR imaging of nitric |
| 1,4:3,6-Dianhydro-D-glucitol | oxide production from isosorbide dinitrate in mice. Am J |
| dinitrate | Physiol. 274: G857-62 (1998). |
| Glyceryl trinitrate: | Benjamin, N., et al., Human vascular smooth muscle cells |
| 1,2,3-Propanetriol trinitrate | inhibit platelet aggregation when incubated with glyceryl |
| | trinitrate: evidence for generation of nitric oxide. Br J |
| | Pharmacol. 102: 847-50 (1991). |
| S-nitroso-glutathione | Heuer, O., et al., Effects of various nitric oxide donating agents |
| | on the contractility and cyclic nucleotide turnover of human |
| | seminal vesicles in vitro. Urology. 59: 958-62 (2002). |
| S-nitroso-N- | Heuer, O., et al., Effects of various nitric oxide donating agents |
| acetylcysteineetylester | on the contractility and cyclic nucleotide turnover of human |
| | seminal vesicles in vitro. Urology. 59: 958-62 (2002). |
| S-nitroso-N-acetylpencillamine | Ulibarri, J. A., et al., Nitric oxide donors, sodium nitroprusside |
| | and S-nitroso-N-acetylpencillamine, stimulate myoblast |
| | proliferation in vitro. In Vitro Cell Dev Biol Anim. 35: 215-8 |
| | (1999). |
| Linsidomine: | Lablanche, J. M., et al., Effect of the direct nitric oxide donors |
| 3-Morpholinosydnone imine | linsidomine and molsidomine on angiographic restenosis after |
| | coronary balloon angioplasty. The ACCORD Study. |
| | Angioplastic Coronaire Corvasal Diltiazem. Circulation. 95: 83-9 |
| | (1997). |
| Molsidomine: | Lablanche, J. M., et al., Effect of the direct nitric oxide donors |
| N-Ethoxycarbonyl-3- | linsidomine and molsidomine on angiographic restenosis after |
| morpholinylsydnoneimine | coronary balloon angioplasty. The ACCORD Study. |
| | Angioplastic Coronaire Corvasal Diltiazem. Circulation. 95: 83-9 |
| | (1997). |
| Mesoionic oxatriazoles | Gryglewski R. J., et al., Mesoionic oxatriazoles (MOTA): NO- |
| | donating characteristics and pharmacology. Curr Pharm Des. |
| | 8: 167-76 (2002). |
| Sodium nitrite | Kohno, M., et al., ESR demonstration of nitric oxide production |
| | from nitroglycerin and sodium nitrite in the blood of rats. Free |
| | Radic Biol Med. 18: 451-7 (1995). |
| FK409: | Kita, Y., et al., Comparison of hemodynamic effects of nitric |
| (+/−)-(E)-ethyl-2-[(E)-hydroxyimino]- | oxide (NO) donors with different NO-releasing properties in |
| 5-nitro-3-hexenamide | rats. J Cardiovasc Pharmacol. 30: 223-8 (1997). |
| FR 146801: | Kita, Y., et al., Comparison of hemodynamic effects of nitric |
| (+/−)-N-[(E)-4-ethyl-3-[(Z)- | oxide (NO) donors with different NO-releasing properties in |
| hydroxyimino]-6-methyl-5-nitro-3- | rats. J Cardiovasc Pharmacol. 30: 223-8 (1997). |
| heptenyl]-3-pyridinecarboxamide | |
| CHF 2206: | Civelli, M., et al., CHF 2206, a new potent vasodilating and |
| 3,4-disubstituted furoxan | antiaggregating drug as potential nitric oxide donor. Eur J |
| | Pharmacol. 255: 17-24 (1994). |
| CHF 2363: | Civelli, M., et al., The involvement of the release of nitric oxide |
| 4-ethoxy-3-phenylsulphonylfuroxan | in the pharmacological activity of the new furoxan derivative |
| | CHF 2363. Br J Pharmacol. 118: 923-8 (1996). |
| R-substituted and di-R- | Ferioloi, R., et al., A new class of furoxan derivatives as NO |
| substituted phenylfuroxans | donors: mechanism of action and biological activity. Br J |
| | Pharmacol. 114: 816-20 (1995). |
| N- and S-nitrooxypivaloyl- | Kartasasmita, R. E., et al., NO-donors (VII [1]): synthesis and |
| cysteine derivatives of naproxen | cyclooxygenase inhibitory properties of N- and S- |
| | nitrooxypivaloyl-cysteine derivatives of naproxen —a novel type |
| | of NO-NSAID. Arch Pharm (Weinheim). 335: 363-6 (2002). |
| Diazeniumdiolates | Megson, I. L., Nitric oxide donor drugs. Drugs of the Future. |
| | 25: 701-15 (2000). |
| JS-K: | Shami P. J., et al., JS-K, a Glutathione/Glutathione S- |
| O(2)-(2,4-Dinitrophenyl) 1-[(4- | Transferase-activated Nitric Oxide Donor of the |
| ethoxycarbonyl) piperazin-1- | Diazeniumdiolate Class with Potent Antineoplastic Activity. Mol |
| yl]diazen-1-ium-1,2-diolate | Cancer Ther. 2: 409-17 (2003). |
| MAHMA NONOate: | Homer, K. L. and Wanstall, J. C. Inhibition of rat platelet |
| (Z-1-N-methyl-N-[6-(N- | aggregation by the diazeniumdiolate nitric oxide donor MAHMA |
| methylammoniohexyl)amino]diazen- | NONOate. Br J Pharmacol. 137: 1071-81 (2002). |
| 1-ium-1,2-diolate | |
| V-PYRRO/NO: | Stinson, S. F., et al., Plasma pharmacokinetics of a liver- |
| O(2)-vinyl 1-(pyrrolidin-1-yl)diazen- | selective nitric oxide-donating diazeniumdiolate in the male |
| 1-ium-1,2-diolate | C57BL/6 mouse. Xenobiotica. 32: 339-47 (2002). |
| 1,3-(nitrooxymethyl)phenyl 2- | U.S. Pat. No. 6,538,033 to Bing |
| hydroxybenzoate | |
| Amidine- and enamine-derived | U.S. Pat. Nos. 6,232,336 and 6,511,991 to Hrabie, et al. |
| diazeniumdiolates | |
| Compounds containing | U.S. Pat. No. 6,492,405 to Haj-Yehia |
| sulfhydryl groups and NO donor | |
| groups | |
| Chitosan-based polymeric nitric | U.S. Pat. Nos. 6,261,594 and 6,451,337 to Smith et al. |
| oxide donors | |

The method and combination of the present invention are useful for, but not limited to, the prevention or treatment of pain and inflammation in a subject, and for treatment of inflammation-related disorders, such as for use as an analgesic in the treatment of pain and headaches, or as an antipyretic for the treatment of fever. For example, combinations of the invention would be useful as anti-inflammatory agents to treat arthritis, including, but not limited to, rheumatoid arthritis, spondyloarthopathies, gouty arthritis, osteoarthritis, systemic lupus erythematosus and juvenile arthritis. Such combinations of the invention would be useful in the treatment of asthma, bronchitis, menstrual cramps, tendonitis, bursitis, connective tissue injuries or disorders, and skin related conditions such as psoriasis, eczema, burns and dermatitis.

In other preferred embodiments, the methods and compositions of the present invention encompass the prevention or treatment of several inflammation-related disorders selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In still other preferred embodiments, the methods and compositions of the present invention encompass the prevention and treatment of the connective tissue and joint disorders selected from the group consisting of arthritis, rheumatoid arthritis, spondyloarthopathies, gouty arthritis, carpal tunnel syndrome, canine hip dysplasia, systemic lupus erythematosus, osteoarthritis, tendonitis and bursitis.

In other preferred embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In other preferred embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, cardiac damage, diabetic cardiac myopathy, renal insufficiency, renal injury, renal arteriopathy, peripheral vascular disease, left ventricular hypertrophy, cognitive dysfunction, stroke, and headache.

In other preferred embodiments, the methods and compositions of the present invention encompass the prevention and treatment of the metabolic disorders selected from the group consisting of obesity, overweight, type I and type II diabetes, hypothyroidism, and hyperthyroidism.

In other preferred embodiments, the methods and compositions of the present invention encompass the prevention and treatment of the respiratory disorders selected from the group consisting of asthma, bronchitis, chronic obstructive pulmonary disease (COPD), cystic fibrosis, pulmonary embolism, pneumonia, pulmonary fibrosis, respiratory failure, acute respiratory distress syndrome and emphysema.

In other preferred embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other preferred embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, influenza, warts, oral herpes, genital herpes, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In other preferred embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In other preferred embodiments, the methods and compositions of the present invention encompass the prevention and treatment of the dermatological disorders selected from the group consisting of acne, psoriasis, eczema, burns, poison ivy, poison oak and dermatitis.

In other preferred embodiments, the methods and compositions of the present invention encompass the prevention and treatment of the surgical disorders selected from the group consisting of pain and swelling following surgery, infection following surgery and inflammation following surgery.

In other preferred embodiments, the methods and compositions of the present invention encompass the prevention and treatment of the gastrointestinal disorders selected from the group consisting of inflammatory bowel syndrome, Crohn's disease, gastritis, irritable bowel syndrome, diarrhea, constipation, dysentery, ulcerative colitis, gastric esophageal reflux, ulcers, and heartburn.

In other preferred embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In other preferred embodiments, the methods and compositions of the present invention encompass the prevention and treatment of the ophthalmic disorders selected from the group consisting of conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

Combinations of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Combinations of the invention would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

Compositions having the novel combination would also be useful in the treatment of ophthalmic diseases, such as retinitis, retinopathies, conjunctivitis, uveitis, ocular photophobia, and of acute injury to the eye tissue. The compositions would also be useful in the treatment of pulmonary inflammation, such as that associated with viral infections and cystic fibrosis. The compositions would also be useful for the treatment of certain central nervous system disorders such as cortical dementias including Alzheimer's disease.

As used herein, the terms “inflammation or inflammation-related disorder”, and “Cox-2 mediated disorder” are meant to include, without limitation, each of the symptoms or diseases that is mentioned above.

The combination of the Cox-2 selective inhibitor and the nitric oxide-donating agent can be supplied in the form of a novel composition that is believed to be within the scope of the present invention. The relative amounts of each component in the composition may be varied and may be as described just above. The Cox-2 selective inhibitor and the nitric oxide-donating agent can be provided in the composition so that the preferred amounts of each of the components are supplied by a single dosage, a single injection or a single capsule for example, or, by up to four, or more, single dosage forms. In preferred embodiments, the composition comprising a Cox-2 selective inhibitor and a nitric oxide-donating agent is useful for treating or preventing a cardiovascular disorder in a subject in need of such prevention or treatment.

When the novel combination is supplied along with a pharmaceutically acceptable excipient, or carrier (which, as used herein, mean the same thing), a pharmaceutical composition is formed. The pharmaceutical composition comprises a pharmaceutically acceptable carrier, a Cox-2 selective inhibitor, and a nitric oxide-donating agent. The pharmaceutical composition of the present invention is directed to a composition suitable for the treatment or prevention of a cardiovascular disorder.

Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The exact dosage and regimen for administering a Cox-2 inhibitor in combination with a nitric oxide-donating agent will necessarily depend upon the potency and duration of action of the compounds used, the nature and severity of the illness to be treated, as well as the sex, age, weight, general health, and individual responsiveness of the subject to be treated, and other relevant circumstances. Those skilled in the art will appreciate that dosages may also be determined with guidance from Goodman & Goldman's The Pharmacological Basis of Therapeutics, Ninth Edition (1996), Appendix II, pp. 1707-1711.

The appropriate dosage level of a Cox-2 inhibitor will generally be from about 0.01 mg per kg to about 140 mg per kg subject body weight per day, which may be administered in single or multiple doses. Preferably, the dosage level will be about 0.1 mg/kg to about 25 mg/kg per day; more preferably about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, preferably once or twice per day.

The amount of the Cox-2 inhibitor that may be combined with carrier materials to produce a single dosage form will vary depending upon the subject treated and the particular mode of administration. For example, a formulation intended for oral administration to humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient.

The dosage level of a nitric oxide-donating agent will necessarily depend on the particular nitric oxide-donating agent that is used. However, in general, the appropriate dosage level of a nitric oxide-donating agent will generally be from about 0.0001 mg per kg to about 200 mg per kg subject body weight per day, which may be administered in single or multiple doses. Preferably, the dosage level will be about 0.001 mg per kg to about 100 mg per kg per day; more preferably about 0.01 mg per kg to about 50 mg per kg per day; even more preferably about 0.1 mg per kg to about 10 mg per kg subject body weight.

A combination therapy comprising a nitric oxide-donating agent that is intended for oral administration to humans may contain from about 10 micrograms to about 10 grams of active agent optionally compounded with an appropriate and convenient amount of carrier material, which may vary from about 5 to about 95 percent of the total composition. More preferably, the nitric oxide-donating agent is dosed at between about 0.1 mg and about 1 gram. Even more preferably, the nitric oxide-donating agent is dosed at between about 1 mg and about 750 mg. More preferably still, the nitric oxide-donating agent is dosed at between about 100 mg and about 500 mg.

For purposes of calculating dosages, it is assumed that the weight of a normal human adult subject is 70 kilograms.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

The phrase “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of the Cox-2 selective inhibitor and the nitric oxide-donating agent. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group Ia) salts, alkaline earth metal (Group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The phrases “combination therapy”, “co-administration”, “administration with”, or “co-therapy”, in defining the use of a Cox-2 selective inhibitor and a nitric oxide-donating agent, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

The pharmaceutical compositions may be administered enterally, parenterally, or topically, such as by inhalation. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The subject method of administering a Cox-2 inhibitor in combination with a nitric oxide-donating agent and compositions comprising the same can also be administered parenterally, either subcutaneously, or intravenously, or intramuscularly, or intrasternally, or by infusion techniques, in the form of sterile injectable aqueous or oleaginous suspensions.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Oral (intra-gastric) is a preferred route of administration for the combination therapy. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Dispersible powders and granules suitable for preparation of an aqueous suspension by the addition of water provide the active ingredients in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the Cox-2 selective inhibitor in combination with the nitric oxide-donating agent may be formulated with sweetening agents, for example glycerol, sorbitol, or sucrose. Such formulations may also contain a demulcent, a preservative and flavoring and coloring agents.

The therapeutic composition containing the Cox-2 selective inhibitor and the nitric oxide-donating agent can be administered by direct inhalation into the respiratory system for delivery as a mist or other aerosol or dry powder. Aerosols of liquid particles comprising the active materials may be produced by any suitable means, such as inhalatory delivery systems. Nebulizers are commercially available devices which transform solutions or suspensions of the active ingredient into a therapeutic aerosol mist either by means of acceleration of compressed gas, typically air or oxygen, through a narrow venture orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One type of solid particulate aerosol generator is an insufflator. Suitable formulations for administration by insufflation include finely comminuted powders which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually-operated pump. The powder employed in the insufflator consists either solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the Cox-2 inhibitor and the nitric oxide-donating agent in a liquified propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

Administration can also be rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

Other methods for administration of the Cox-2 selective inhibitor compound and the nitric oxide-donating agent include dermal patches that release the medicaments directly into a subject's skin.

Topical delivery systems are also encompassed by the present invention and include ointments, powders, sprays, creams, jellies, collyriums, solutions or suspensions.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-1 03), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a Cox-2 selective inhibitor and nitric oxide-donating agent topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins) (2000); Hoover, John E., Remington's Pharmaceutical Sciences, Mack Publishing Co., Easton Pennsylvania (1975); Liberman, et al., Eds., Pharmaceutical Dosage Forms, Marcel Decker, New York, N.Y. (1980); and Kibbe, et al., Eds., Handbook of Pharmaceutical Excipients (3^{rd} Ed.), American Pharmaceutical Association, Washington (1999).

The present invention further comprises kits that are suitable for use in performing the methods of treatment described above. In one embodiment, the kit contains a first dosage form comprising a Cox-2 selective inhibitor and a second dosage form comprising a nitric oxide-donating agent, in quantities sufficient to carry out the methods of the present invention. Preferably, the first dosage form and the second dosage form together comprise a therapeutically effective amount of the compounds for the prevention or treatment of pain, inflammation, and inflammation-related disorders.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### EXAMPLE 1

This example demonstrates the production of 4-(5-Methyl-3-phenylisoxazol-4-yl)benzenesulfonamide (valdecoxib), following the disclosure provided in U.S. Pat. No. 5,633,272.

### Step 1. Preparation of desoxybenzoin keto-oxime

Desoxybenzoin (20.0 g, 0.102 mol) was dissolved in toluene (200 mL). In a separate 500 mL round-bottom flask equipped with a magnetic stirring bar, hydroxylamine hydrochloride (9.21 g, 0.132 mol) and potassium hydroxide (7.43 g, 0.132 mol) were suspended in absolute ethanol (50 mL) and stirred vigorously at room temperature for thirty minutes. The desoxybenzoin solution was added in one portion, and the yellow suspension was held at reflux, using a Dean-Stark trap to remove generated water, under a nitrogen blanket for 16 hours. The suspension was cooled to room temperature and poured into water (200 mL). The system was extracted with ethyl acetate (2×150 mL), then the combined organic solution was washed with brine (200 mL), dried over magnesium sulfate, and filtered. The solvents were evaporated under reduced pressure to yield a crude solid. The solid was recrystallized from hot ethanol/water, filtered and washed with water to yield, upon drying, desoxybenzoin keto-oxime as white crystals (17.7 g, 82%): mp 87°-90° C. Mass spectrum, MH+=212. High resolution mass spectrum Calc'd. for C₁₄ H₁₃ NO: 211.0997. Found: 211.0949.

### Step 2. Preparation of 4-[5-methyl-3-phenylisoxazol-4-yl]benzensulfonamide

A solution of desoxybenzoin keto-oxime from Step 1 (6.00 g; 28.40 mmol) in anhydrous tetrahydrofuran (THF, 80 mL) was cooled to −20° C. in an oven-dried 250 mL three-neck round-bottom flask equipped with a thermometer, nitrogen gas inlet, rubber septum and provisions for magnetic stirring. To this cold solution, n-butyllithium (1.6N in hexanes, 44.4 mL) was added, via syringe, over 35 minutes, such that the reaction temperature remained at or below −10° C. The deep red solution was stirred at −10° C. for 1 hour, warmed to room temperature, then stirred at room temperature for an additional hour. Acetic anhydride (3.2 mL, 34.1 mmol) was added in one portion, and the resulting suspension was stirred without temperature control for 2 hours. Water (100 mL) was added, and the solution was poured into 1N HCl (100 mL) and extracted with ethyl acetate (2×200 mL). The combined organic solution was washed with hydrochloric acid (1N HCl, 100 mL) and brine (100 mL), dried over magnesium sulfate and filtered. The resulting solution was evaporated under reduced pressure to yield a crude oil. The oil was applied to a column of silica gel and eluted with ethyl acetate/hexane (10-50% ethyl acetate) to yield, upon concentration of the appropriate fractions, 5.0 g of 3,4-diphenyl-4-hydrido-5-hydroxy-5-methylisoxazole. The solid was cooled to 0° C., then dissolved in cold chlorosulfonic acid (15 mL). The brown solution was stirred at 0° C. for 2 hours, then added dropwise to a stirring suspension of ice (200 mL) and dichloromethane (200 mL). The layers were separated, and the organic phase was added directly to a saturated ammonium hydroxide solution (100 mL) at 0° C. This biphasic solution was vigorously stirred at 0° C. for 2 hours, the layers were separated, and the aqueous phase was washed with dichloromethane (50 mL). The combined organic solution was dried over magnesium sulfate, filtered and evaporated under reduced pressure to approximately one-half of its original volume. Crystals formed. The stirred suspension was cooled to 0° C. and held for 30 minutes. The crystals were filtered, washed with cold dichloromethane and dried to yield 4-[5-methyl-3-phenylisoxazol-4-yl]benzenesulfonamide (2.7 g, 30%): mp 155.degree.-157.degree. C. ¹H NMR (CD₃ CN/500 MHz) Δ 7.86 (d, J=8.39 Hz, 2H), 7.45 (m, 1H), 7.39 (s, 4H, 7.37 (d, J=8.39 Hz, 2H), 5.70 (s, 2H), 2.46 (s, 3H Mass Spectrum, MH+=315).

### EXAMPLE 2

This example illustrates the production of a composition containing valdecoxib and the NO-donating agent isosorbide dinitrate, and of a pharmaceutical composition containing the combination.

Valdecoxib can be prepared as described in Example 1 or, alternatively, can be obtained under the trade name Bextra® from Pfizer, Inc., New York, N.Y.

Isosorbide dinitrate can be obtained under the trade name Isordil® from Wyeth-Ayerst, Madison, N.J.

A therapeutic composition of the present invention can be produced by intermixing finely powdered isosorbide dinitrate (20 g, as available from Wyeth-Ayerst, Madison, N.J., under the trade name Isordil®) and valdecoxib (10 g, as produced in Example 1, or as available from Pfizer, Inc., New York, N.Y., under the tradename Bextra®) in a laboratory mill or mixing device suitable for mixing of powders without generating shear force or temperature sufficient to degrade either of the two compounds.

After mixing, the combination of valdecoxib and isosorbide dinitrate forms a therapeutic composition that is sufficient for the production of 1000 human single dose unit, each dose containing 10 mg of valdecoxib and 20 mg of isosorbide dinitrate.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

1. A method of preventing or treating pain, inflammation, or inflammation-related disorders in a subject in need of such prevention or treatment by administering to the subject a Cox-2 selective inhibitor in combination with a nitric oxide-donating agent.

2. The method according to claim 1, wherein the amount of the Cox-2 selective inhibitor and the amount of the nitric oxide-donating agent together constitute an amount effective for the treatment or prevention of the pain, inflammation or inflammation-related disorder.

3. The method according to claim 1, wherein the Cox-2 selective inhibitor comprises a chromene Cox-2 selective inhibitor.

4. The method according to claim 3, wherein the chromene Cox-2 selective inhibitor is selected from the group consisting of substituted benzothiopyrans, dihydroquinolines, or dihydronaphthalenes having the general formula: 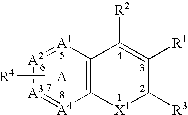
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b} R^{c} forms a 3-6 membered cycloalkyl ring;
wherein R¹ is selected from carboxyl, aminocarbonyl, C₁-C₆-alkylsulfonylaminocarbonyl and C₁-C₆-alkoxycarbonyl;
wherein R² is selected from hydrido, phenyl, thienyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, cyano and cyano-C₁-C₃-alkyl;
wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R⁴ together with ring A forms a radical selected from naphthyl, quinolyl, isoquinolyl, quinolizinyl, quinoxalinyl and dibenzofuryl;
or an isomer or pharmaceutically acceptable salt thereof.

5. The method according to claim 3, wherein the chromene Cox-2 selective inhibitor is selected from the group consisting of substituted benzothiopyrans, dihydroquinolines, or dihydronaphthalenes having the general formula: 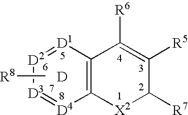
wherein X² is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R⁵ is selected from carboxyl, aminocarbonyl, C₁-C₆-alkylsulfonylaminocarbonyl and C₁-C₆-alkoxycarbonyl;
wherein R⁶ is selected from hydrido, phenyl, thienyl, C₂-C₆-alkynyl and C₂-C₆-alkenyl;
wherein R⁷ is selected from C₁-C₃-perfluoroalkyl, chloro, C₁-C₆-alkylthio, C₁-C₆-alkoxy, nitro, cyano and cyano-C₁-C₃-alkyl;
wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the D ring atoms D¹, D², D³ and D⁴ are independently selected from carbon and nitrogen with the proviso that at least two of D¹, D², D³ and D⁴ are carbon;
or wherein R⁸ together with ring D forms a radical selected from naphthyl, quinolyl, isoquinolyl, quinolizinyl, quinoxalinyl and dibenzofuryl;
or an isomer or pharmaceutically acceptable salt thereof.

6. The method according to claim 3, wherein the chromene Cox-2 selective inhibitor is selected from the group consisting of substituted benzothiopyrans, dihydroquinolines, or dihydronaphthalenes having the general formula: 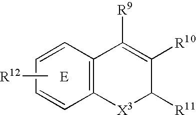
wherein X³ is selected from the group consisting of O or S or NR^{a};
wherein R^{a} is alkyl;
wherein R⁹ is selected from the group consisting of H and aryl;
wherein R¹⁰ is selected from the group consisting of carboxyl, aminocarbonyl, alkylsulfonylaminocarbonyl and alkoxycarbonyl;
wherein R¹¹ is selected from the group consisting of haloalkyl, alkyl, aralkyl, cycloalkyl and aryl optionally substituted with one or more radicals selected from alkylthio, nitro and alkylsulfonyl; and
wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or
wherein R¹² together with ring E forms a naphthyl radical; or an isomer or pharmaceutically acceptable salt thereof;
and including the diastereomers, enantiomers, racemates, tautomers, salts, esters, amides and prodrugs thereof.

7. The method according to claim 3, wherein the chromene Cox-2 selective inhibitor comprises a compound having the formula: 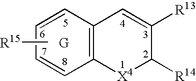
wherein X⁴ is selected from O or S or NR^{a};
wherein R^{a} is alkyl;
wherein R¹³ is selected from carboxyl, aminocarbonyl, alkylsulfonylaminocarbonyl and alkoxycarbonyl;
wherein R¹⁴ is selected from haloalkyl, alkyl, aralkyl, cycloalkyl and aryl optionally substituted with one or more radicals selected from alkylthio, nitro and alkylsulfonyl; and
wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R¹⁵ together with ring G forms a naphthyl radical;
or an isomer or pharmaceutically acceptable salt thereof.

8. The method according to claim 3, wherein the chromene Cox-2 selective inhibitor comprises a compound having the formula: 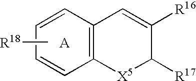
wherein:
X⁵ is selected from the group consisting of O or S or NR^{b};
R^{b} is alkyl;
R¹⁶ is selected from the group consisting of carboxyl, aminocarbonyl, alkylsulfonylaminocarbonyl and alkoxycarbonyl;
R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R¹⁸ together with ring A forms a naphthyl radical;
or an isomer or pharmaceutically acceptable salt thereof.

9. The method according to claim 8, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of lower haloalkyl, lower cycloalkyl and phenyl; and
R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R¹⁸ together with ring A forms a naphthyl radical;
or an isomer or pharmaceutically acceptable salt thereof.

10. The method according to claim 8, wherein:
R¹⁶ is carboxyl;
R¹⁷ is lower haloalkyl; and
R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl;
or wherein R¹⁸ together with ring A forms a naphthyl radical;
or an isomer or pharmaceutically acceptable salt thereof.

11. The method according to claim 8, wherein:
R¹⁶ is selected from the group consisting of carboxyl, lower alkyl, lower aralkyl and lower alkoxycarbonyl;
R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl;
or wherein R² together with ring A forms a naphthyl radical;
or an isomer or pharmaceutically acceptable salt thereof.

12. The method according to claim 8, wherein:
R¹⁶ is selected from the group consisting of carboxyl, lower alkyl, lower aralkyl and lower alkoxycarbonyl;
R¹⁷ is selected from the group consisting trifluoromethyl and pentafluoroethyl; and
R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl;
or wherein R¹⁸ together with ring A forms a naphthyl radical;
or an isomer or prodrug thereof.

13. The method according to claim 3, wherein the chromene Cox-2 selective inhibitor comprises a compound having the formula: 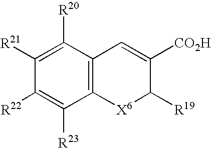
wherein:
X⁶ is selected from the group consisting of O and S;
R¹⁹ is lower haloalkyl;
R²⁰ is selected from the group consisting of hydrido, and halo;
R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R²² is selected from the group consisting of hydrido, lower alkyl, halo, lower alkoxy, and aryl; and
R²³ is selected from the group consisting of the group consisting of hydrido, halo, lower alkyl, lower alkoxy, and aryl;
or an isomer or prodrug thereof.

14. The method according to claim 13, wherein:
X⁶ is selected from the group consisting of O and S;
R¹⁹ is selected from the group consisting of trifluoromethyl and pentafluoroethyl;
R²⁰ is selected from the group consisting of hydrido, chloro, and fluoro;
R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R²² is selected from the group consisting of hydrido, methyl, ethyl, isopropyl, tert-butyl, chloro, methoxy, diethylamino, and phenyl; and
R²³ is selected from the group consisting of hydrido, chloro, bromo, fluoro, methyl, ethyl, tert-butyl, methoxy, and phenyl;
or an isomer or prodrug thereof.

15. The method according to claim 3, wherein the chromene Cox-2 selective inhibitor is selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

16. The method according to claim 1, wherein the Cox-2 selective inhibitor is a tricyclic Cox-2 selective inhibitor selected from the group consisting of valdecoxib, rofecoxib, JTE-522, parecoxib, and pharmaceutically acceptable salts and prodrugs thereof.

17. The method according to claim 1, wherein the Cox-2 selective inhibitor is selected from the group consisting of valdecoxib and pharmaceutically acceptable salts and prodrugs thereof.

18. The method according to claim 1, wherein the Cox-2 selective inhibitor comprises a phenylacetic acid derivative represented by the general structure: 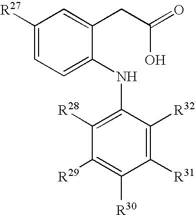
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxy;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H;
or a prodrug thereof.

19. The method according to claim 18, wherein:
R²⁷ is ethyl;
R²⁸ and R³⁰ are chloro;
R²⁹ and R³¹ are hydrogen; and
R³² is methyl;
or a prodrug thereof.

20. The method according to claim 18, wherein:
R²⁷ is propyl;
R²⁸ and R³⁰ are chloro;
R²⁹ and R³¹ are methyl; and
R³² is ethyl;
or a prodrug thereof.

21. The method according to claim 18, wherein:
R²⁷ is methyl;
R²⁸ is fluoro;
R³² is chloro; and
R²⁹, R³⁰, and R³¹ are hydrogen;
or a prodrug thereof.

22. The method according to claim 1, wherein the Cox-2 selective inhibitor comprises a compound having the general formula: 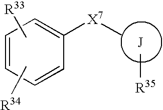
wherein:
X⁷ is O; J is 1-phenyl; R³³ is 2-NHSO₂CH₃; R³⁴ is 4-NO₂; and there is no R³⁵ group, (nimesulide), and
X⁷ is O; J is 1-oxo-inden-5-yl; R³³ is 2-F; R³⁴ is 4-F; and R³⁵ is 6-NHSO₂CH₃, (flosulide); and
X⁷ is O; J is cyclohexyl; R³³ is 2-NHSO₂CH₃; R³⁴ is 5-NO₂; and there is no R³⁵ group, (NS-398); and
X⁷ is S; J is 1-oxo-inden-5-yl; R³³ is 2-F; R³⁴ is 4-F; and R³⁵ is 6-N⁻SO₂CH₃.Na⁺,
(L-745337); and
X⁷ is S; J is thiophen-2-yl; R³³ is 4-F; there is no R³⁴ group; and R³⁵ is 5-NHSO₂CH₃, (RWJ-63556); and
X⁷ is O; J is 2-oxo-5I-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

23. The method according to claim 22, wherein the phenylacetic acid derivative Cox-2 selective inhibitor is lumiracoxib.

24. The method according to claim 1, wherein the Cox-2 selective inhibitor comprises a diarylmethylidenefuran derivative.

25. The method according to claim 1, wherein the nitric oxide-donating agent is selected from the group consisting of pirsidomine, sodium nitroprusside, isorbide dinitrate, glyceryl trinitrate, S-nitroso-glutathione, S-nitroso-N-acetylcysteineetylester, S-nitroso-N-acetylpencillamine, linsidomine, molsidomine, mesoionic oxatriazoles, sodium nitrite, FK409, FR 146801, CHF 2206, CHF 2263, R-substituted and di-R-substituted phenylfuroxans, N- and S-nitrooxypivaloyl-cysteine derivatives of naproxen, diazeniumdiolates, JS-K, MAHMA NONOate, V-PYRRO/NO, and combinations thereof.

26. The method according to claim 1, wherein the inflammation or inflammation-related disorder is selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

27. The method according to claim 26, wherein the cardiovascular disorder is selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis arteriosclerosis, atherosclerosis, vascular and valvar calcification, coronary artery disease, heart failure, congestive heart failure, shock, arrhythmia, left ventricular hypertrophy, angina, diabetic nephropathy, kidney failure, eye damage, cardiac damage, diabetic cardiac myopathy, renal insufficiency, renal injury, renal arteriopathy, peripheral vascular disease, left ventricular hypertrophy, cognitive dysfunction, stroke, and headache.

28. The method according to claim 26, wherein the connective tissue and joint disorder is selected from the group consisting of arthritis, rheumatoid arthritis, spondyloarthopathies, gouty arthritis, carpal tunnel syndrome, canine hip dysplasia, systemic lupus erythematosus, osteoarthritis, tendonitis and bursitis.

29. The method according to claim 26 wherein the connective tissue and joint disorder is osteoarthritis.

30. The method according to claim 26 wherein the connective tissue and joint disorder is rheumatoid arthritis.

31. A therapeutic composition comprising a Cox-2 selective inhibitor and a nitric oxide-donating agent.

32. A pharmaceutical composition comprising a Cox-2 selective inhibitor, a nitric oxide-donating agent, and a pharmaceutically acceptable excipient.

33. A kit that is suitable for use in the treatment or prevention of pain, inflammation, and inflammation-related disorders, the kit comprising a first dosage form comprising a Cox-2 selective inhibitor and a second dosage form comprising a nitric oxide-donating agent, in quantities which comprise a therapeutically effective amount of the combination of the compounds for the treatment or prevention of pain, inflammation, or inflammation-related disorders.
